SEQUENCE LISTING (27 sequences)

SEQ ID NO: 1 - DNA, 274 bp, Homo sapiens
ctcctgtctcagcctccctagtagatgggattacaggcgtgcaccaccacgcccagcccagttaatttttgtatttttagacagacagggtttcaccatgttggtcaggctggtcttgaactcctgatctcagatgatgcacctggctcggcctcgcaaagtgctgggattacaggtgtgagccaccgtgcccagccgttagctcattttaacacatccttagtccagcctgttccaaaaaatctaaagtcagatagcttcctaaacctcaact

SEQ ID NO: 2 - DNA, 20 bp, Artificial
  Nanog-FWD primer
ctcctgtctcagcctcccta

SEQ ID NO: 3 - DNA, 24 bp, Artificial
  Nanog-REV short primer
agttgaggtttaggaagctatctg

SEQ ID NO: 4 - DNA, 19 bp, Artificial
  AT adapter oligonucleotide
gtaaaacgacggccagtat

SEQ ID NO: 5 - DNA, 17 bp, Artificial
  13(-20)-REV primer
actggccgtcgttttac

SEQ ID NO: 6 - DNA, 71 bp, Artificial
  real time PCR detection product from Nanog promoter
ctgggattacaggtgtgagccaccgtgcccagccgttagctcattttaacacatactggccgtcgttttac

SEQ ID NO: 7 - DNA, 19 bp, Artificial
  Detection primer
ctgggattacaggtgtgag

SEQ ID NO: 8 - DNA, 17 bp, Artificial
  M13(-20) primer
gtaaaacgacggccagt

SEQ ID NO: 9 - DNA, 24 bp, Artificial
  primer for amplifying T4 DNA
gtgaagtaagtaataaatggattg

SEQ ID NO: 10 - DNA, 24 bp, Artificial
  primer for amplifying T4 DNA
tggagaaggagaatgaagaataat

SEQ ID NO: 11 - DNA, 27 bp, Artificial
  primer for amplifying T4 DNA
gccatattgataatgaaattaaatgta

SEQ ID NO: 12 - DNA, 26 bp, Artificial
  primer for amplifying T4 DNA
tcagcaattttaatatttccatcttc

SEQ ID NO: 13 - DNA, 20 bp, Artificial
  primer for amplifying T4 DNA
tatactgaagtacttcatca

SEQ ID NO: 14 - DNA, 20 bp, Artificial
  primer for amplifying T4 DNA
ctttgcgtgatttatatgta

SEQ ID NO: 15 - DNA, 39 bp, Artificial
  primer for amplifying T4 DNA
ctcgtagactgcgtaccaatctaactcaggatagttgat

SEQ ID NO: 16 - DNA, 22 bp, Artificial
  primer for amplifying T4 DNA
tatgataagtatgtaggttatt

SEQ ID NO: 17 - DNA, 18 bp, Artificial
  primer for amplifying T4 DNA
ctcgtagactgcgtacca

SEQ ID NO: 18 - DNA, 25 bp, Artificial
  primer for amplifying T4 DNA
tatgataagtatgtaggttattcaa

SEQ ID NO: 19 - DNA, 21 bp, Artificial
  primer for amplifying nanog DNA
tcaggagtttgggaccagcta

SEQ ID NO: 20 - DNA, 18 bp, Artificial
  primer for amplifying nanog DNA
cccccctcaagcctccta

SEQ ID NO: 21 - DNA, 22 bp, Artificial
  linker oligonucleotide
ctcgtagactgcgtaccatgnn

SEQ ID NO: 22 - DNA, 20 bp, Artificial
  linker oligonucleotide
catggtacgcagtctaccag

SEQ ID NO: 23 - DNA, 20 bp, Artificial
  primer for amplifying digestion products from T4 DNA substrates
ctcgtagactgcgtaccatg

SEQ ID NO: 24 - DNA, 21 bp, Artificial
  primer for amplifying digestion products from T4 DNA
gagtcagaccttgctgccaaa

SEQ ID NO: 25 - DNA, 21 bp, Artificial
  primer for amplifying digestion products from T4 DNA
gccgtctaagcaatggaagaa

SEQ ID NO: 26 - DNA, 22 bp, Artificial
  primer for amplifying T4 DNA
tatgataagtatgtaggttatt

SEQ ID NO: 27 - DNA, 22 bp, Artificial
  Pvu TRS1I recognition sequence
cnnnnnnnnnnnnnnnnnnnng

(19) United States
(12) Patent Application Publication
(10) Pub. No.: US 2014/0178873 A1
(43) Pub. Date: Jun. 26, 2014

(54) NOVEL METHODS FOR DETECTING HYDROXYMETHYLCYTOSINE

(71) Applicants: Andreas Brachmann, Muenchen (DE); Heinrich Leonhardt, Muenchen (DE); Fabio Spada, Muenchen (DE); Aleksandra Szwagierczak, Muenchen (DE); Christine Silvia Schmidt, Muenchen (DE); Sebastian Bultmann, Olching (DE)

(72) Inventors: Andreas Brachmann, Muenchen (DE); Heinrich Leonhardt, Muenchen (DE); Fabio Spada, Muenchen (DE); Aleksandra Szwagierczak, Muenchen (DE); Christine Silvia Schmidt, Muenchen (DE); Sebastian Bultmann, Olching (DE)

(21) Appl. No.: 14/003,203

(22) PCT Filed: Mar. 2, 2012
(86) PCT No.: PCT/EP2012/053641

§ 371 Date: Mar. 10, 2014

(30) Foreign Application Priority Data
Mar. 4, 2011 (EP) 11001842.1

(51) Int. Cl. C12Q 1/68

(52) U.S. Cl. CPC C12Q 1/6858; C12Q 1/6886; C12Q 1/6883
USPC 435/6.11

(57) ABSTRACT

The present invention provides a method of detecting a hydroxymethyl (hm) cytosine (C) in a nucleic acid molecule preparation; comprising: (a) providing a single-stranded (ss) nucleic acid molecule; (b) synthesizing at least one copy of at least a portion of the complementary strand of said ss nucleic acid molecule thereby generating a double-stranded (ds) nucleic acid molecule, wherein said synthesis is carried out in the presence of hydroxymethylcytosine or analog thereof (e.g., protected hydroxyl group); and (c) reacting the product obtained in (b) (all or purified) with an endonuclease being capable of cleaving said ds nucleic acid molecule, wherein cleavage by said endonuclease requires a recognition site that contains hmC on opposite strands; and (d) analyzing the product obtained in step (c).

In higher eukaryotes only the C5 position of genomic cytosine is subject to enzymatically catalyzed postreplicative modification. Methylation at this position has long been known to play major roles in epigenetic control of transcriptional activity and, as a consequence, to affect fundamental processes such as development (including natural reprogramming of cell fate), imprinting, X chromosome inactivation, genome stability and redisposition to neoplastic transformation (1,2).

The recent discovery of the further modification of 5-methylcytosine (mC) to 5-hydroxymethylcytosine (hmC) by the family of Tet dioxygenases has raised major questions on the functional relevance of this 6th base in mammalian genomes (3,4). While recent evidence supports a role for hmC as an intermediate in the erasure of cytosine methylation (5), other roles in controlling genomic functions cannot be excluded.

The definition of these roles will require profiling of genomic hmC patterns, which presents a major technical challenge as hmC is structurally and chemically very similar to mC but in general far less abundant in mammalian genomes (3,4,6-9).

The gold standard methodology for profiling of genomic mC sites, bisulfite conversion, cannot discriminate hmC from mC and all available restriction endonucleases are either equally sensitive to mC and hmC or not sensitive to either (10-12).

While antibodies raised against hmC are commercially available their use to probe hmC frequency by DNA immunoprecipitation has yet to be reported and the accuracy of this method will depend on the relative affinity of these antibodies for hmC versus mC as the latter is present in large excess in mammalian genomes. Very recently enzymatic methods for selective labeling and identification of hmC have been reported (7,13).

However, prior art methods are merely capable of providing, so to say, a generic landscape view, but are not capable of providing specific information as to which C within a nucleotide sequence of interest is hydroxymethylated. Indeed, Ku et al. (J Med Genet. (2011) 48(11): 721-730) attest to this deficiency of the prior art by pointing out that although next generation sequencing is available, it is not yet possible to distinguish 5-methylcytosine and 5-hydroxymethylcytosine in order to study their biological roles. Similarly, though Matarese et al. (Mol Syst Biol. 2011 (7): 562. doi: 10.1038/msb.2011.95.) describe various methods for detecting and identifying 5-hydroxymethylcytosine in nucleic acid sequences, it is not yet possible to precisely map these modified bases so as to elucidate their function, since thus far available methods only detect and quantitate 5hmC levels, but can neither determine their precise position in the genome nor as to whether they are present on the sense, antisense or both strands.

The same is true for WO 2011/025819. Specifically, while methods for globally detecting hydroxymethylated nucleotides in a nucleotide sequence of interest are presented by, for example, applying restriction enzymes of the so-called weirdo group of restriction endonucleases, which cut hydroxymethylated DNA, these methods do not enable the precise mapping of hydroxymethylated nucleotides. This is so because no teaching is provided on the mode of action (including their recognition sequence) of restriction endonucleases of the weirdo group of restriction endonucleases to which, for example, PvuRts1I belongs, while in particular knowledge of the recognition sequence, more particularly knowledge as to whether such an enzyme requires a hemi- or full-hydroxymethylated recognition sequence would be the first step in order to perhaps exploit that knowledge for further purposes such as the mapping of hydroxymethylated nucleotides. Secondly, even if, on the basis of WO 2011/025819, one would have elucidated the mode of action of a restriction endonuclease that cuts hydroxymethylated DNA, no teaching is provided in that document as how to exploit that mode of action for the purpose of mapping hydroxymethylated nucleotides in a nucleotide sequence of interest.

Accordingly, the technical problem of the present invention is to comply with the needs described above.

### DETAILED DESCRIPTION

The present invention addresses these needs and thus provides as a solution to the technical problem the embodiments concerning methods and means for detecting a hydroxymethyl (hm) cytosine (C) in a nucleic acid molecule preparation as described herein. These embodiments are characterized and described herein, illustrated in the Examples, and reflected in the claims.

Several modification and restriction systems have evolved as defense and counter defense strategies in the struggle between unicellular microorganisms and their viruses. The present invention shows that, in contrast to previously characterized endonucleases which cleave ^{hm}C-containing sequences, PvuRts1I has a preference for the non-glucosylated form of this base and discriminates against ^{m}C. This specificity makes PvuRts1I an attractive tool to investigate genomic ^{hm}C patterns in higher eukaryotes and complements the very recently published methods for enzymatic labeling of this sixth base (7,13).

Importantly, the present invention shows that the extent of PvuRts1I digestion reflects the relative abundance of ^{hm}C in genomic DNA from cerebellum and TKO ESCs. The limited extent of digestion even for samples with relatively high hmC content is in line with the cleavage site preference and dependence on cytosine modification that we determined. We calculate that the statistical probability of the PvuRts1I consensus site CN11-12/N9-10G in the mouse genome is 0.126. Combined with the global hmC occurrence in mouse tissues (up to 0.13% of all bases or 0.65% of Cs) (3, 7-9) this translates into a PvuRts1I cleavage site every 1.9×105 bases. As this is in the size range of fragments typically obtained with standard procedures for isolation of genomic DNA, more careful isolation methods should be used and/or PvuRts1I specific ends could be enriched by ligating biotinylated PvuRts1I compatible linkers.

Alternatively, digestion conditions could be optimized or DNA could be denatured and a second strand synthesized with hmC nucleotides to cut and reveal the likely more abundant hemimodified PvuRts1I sites.

Notably, while cerebellum has been previously reported among the tissues with the highest levels of genomic hmC (3,7,8), complete absence of mC and therefore hmC would be expected in TKO ESCs due to the lack of all three major Dnmts (21). However, it was previously detected that hmC levels are slightly above background in TKO ESCs (7) and the present invention shows minimal but appreciable digestion by PvuRts1I. In this context it is interesting to note that ESCs express the highly conserved Dnmt2 (25,26), the only Dnmt family member with an intact catalytic domain that has not been genetically inactivated in TKO ESCs. Although Dnmt2 has a major role as a tRNA methyltransferase and its function as a DNA methyltransferase is still debated (27-31), it was recently shown to methylate genomic sequences in Drosophila (32,33). Future work should clarify whether the genome of TKO ESCs harbors any residual mC and hmC.

Restriction of genomic DNA with PvuRts1I may be combined with PCR amplification for analysis of specific loci or with massive parallel sequencing or microarray hybridization for genome-wide mapping. The calculations reported above for the frequency of PvuRts1I cleavage sites based on a random hmC distribution bring up the argument that the extent of random breaks in genomic DNA preparations would contribute very significant noise in deep sequencing and microarray applications. This drawback may be at least partially overcome if specific PvuRts1I ends are enriched by ligating linkers with a random two nucleotide 3′ overhang as described here and discussed above, a strategy that can be integrated with procedures for generation of sequencing libraries. Also, as described herein simulation of genomic fragments containing known levels of randomly distributed hmC clearly shows that relatively high local concentrations of hmC sites are required for efficient detection by PvuRts1I.

The first genome-wide hmC profiles from mammalian tissues have just been reported (13). From these first datasets it is apparent that genomic hmC is not randomly distributed and that its accumulation in gene bodies is proportional to transcriptional activity. Thus, PvuRts1I may prove a valuable tool to probe hmC accumulation at defined genomic regions. In addition, the selectivity of PvuRts1I for hmC-containing sites may constitute an advantage with respect to endonucleases such as McrBC and MspJ1 as these enzymes do not discriminate between mC and hmC and require in vitro enzymatic hmC glucosylation to specifically protect hmC-containing sites from digestion and thus distinguish them from mC sites.

In conclusion, the present invention shows that PvuRts1I is an hmC specific endonuclease and provide a biochemical characterization of it enzymatic properties for future applications as diagnostic tools in the analysis of hmC distribution at genomic loci in development and disease.

Accordingly, from the findings of the inventors described herein, it can reasonably be concluded that the present invention envisages that endonucleases of the PvuRts1I family can be applied in the methods and means (in particular kits) of the present invention as described herein.

It must be noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

All publications and patents cited in this disclosure are incorporated by reference in their entirety. To the extent the material incorporated by reference contradicts or is inconsistent with this specification, the specification will supersede any such material.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim.

In each instance herein any of the terms “comprising”, “consisting essentially of” and “consisting of” may be replaced with either of the other two terms.

As used herein, the conjunctive term “and/or” between multiple recited elements is understood as encompassing both individual and combined options. For instance, where two elements are conjoined by “and/or”, a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or” as used herein.

As described herein, “preferred embodiment” means “preferred embodiment of the present invention”. Likewise, as described herein, “various embodiments” and “another embodiment” means “various embodiments of the present invention” and “another embodiment of the present invention”, respectively.

Several documents are cited throughout the text of this specification. Each of the documents cited herein (including all patents, patent applications, scientific publications, manufacturer's specifications, instructions, etc.), whether supra or infra, are

Aspects of the present invention can be summarized in the following items:

(1) A method of detecting a hydroxymethyl (hm) cytosine (C) in a nucleic acid molecule preparation; comprising:
(a) providing a single-stranded (ss) nucleic acid molecule;
(b) synthesizing at least one copy of at least a portion of the complementary strand (for example, by way of a single round amplification) of said ss nucleic acid molecule thereby generating a double-stranded (ds) nucleic acid molecule, wherein said synthesis is carried out in the presence of hydroxymethylcytosine or analog thereof; and
(c) reacting the product obtained in (b) with an endonuclease being capable of cleaving said ds nucleic acid molecule, wherein cleavage by said endonuclease requires a recognition site that contains hmC on opposite strands; and
(d) analyzing the product obtained in step (c).

PvuRts1I was first described by Ishag & Kaji (Biological Chemistry 255(9): 4040-4047 (1980)) and shown to be a hmC-specific restriction endonuclease that is encoded by the plasmid RtsI. The PvuRts1I gene was cloned and expressed (Janosi and Kaji, FASEB J. 6: A216 (1992); Janosi et al. Journal of Molecular Biology 242: 45-61 (1994)) and the RtsI plasmid was completely sequenced (Murata et al., Journal of Bacteriology 184(12): 3194-202 (2002)). However, no in-depth study of this enzyme has been carried out or published. Furthermore, after the initial publications, there has been little interest in this enzyme until the present inventors clarified its recognition sequence and mode of action in order to exploit PvuRts1I and further enzymes of the family to which PvuRts1I belongs for the purposes of the methods of the present invention.

In particular, the present inventors elucidated the recognition sequence of PvuRts1I and, even more importantly, found that PvuRts1I only cleaves a ds nucleic acid molecule, if hmC is present on both strands of said nucleic acid molecule. On the basis of, inter alia, these findings, the present inventors developed an assay that allows to determine as to where (i.e., at which position in a nucleotide of interest) an hmC is present and/or whether an hmC is present on one or both strands (i.e., upper and/or lower strand) by applying an endonuclease being capable of cleaving ds nucleic acid molecules, whereby cleavage by said endonuclease requires a recognition sequence that contains hmC on opposite strands. Said endonuclease is preferably one of the ZZYZ family of restriction endonuclease as described in WO2011/091146.

Accordingly, the present inventors propose to generate a second strand (e.g., either by means and methods for synthesizing a second strand as is known in the art or by oligonucleotide hybridization) that is complementary to a ss nucleic acid molecule of interest (i.e., one which should be inspected for the presence and/or absence of hmC) by using hmC.

Hence, any prior art document such as Swagierczak et al. (cited as “(7)” herein) which provides, e.g., for hmC-containing templates which are substrates for, e.g., PvuRts1I that are generated by nucleic acid amplification are irrelevant, since any nucleic acid amplification for more than one cycle results in products that contain hmC on both strands. However, the methods of the present invention only require the generation of the (complementary) second strand of the ss DNA nucleic acid molecule of interest, since otherwise no analysis of the position of hmCs would be possible. Namely, if, for example, only one strand (e.g., the upper strand) contains hmC, while the lower strand does not, the recognition sequence for the endonuclease is “restored” by the generation of the second strand and, thus, cleavage can occur. However, if no hmC is present in the upper strand, no cleavage would occur, since the recognition sequence would not be restored, because the endonuclease requires hmC on both strands. The same is true for the lower strand. In that case, second strand synthesis of the upper strand is done in the presence of hmC.

In sum, the assay and methods developed by the present inventors pave the way for precisely determining and/or mapping hmCs in a nucleic acid molecule of interest as further detailed herein below.

“Hydroxymethyl (hm) cytosine (C)” as referred to in the method and means of the invention may be modified. The term “modification” here and in the claims refers to a chemical group or biological molecule that is reacted with a hydroxyl group on a nucleotide in a DNA to become attached via a covalent bond.

Modification can be achieved by chemical or enzymatic means. In nature, certain bacterial viruses have modified hydroxymethylated cytosines (mhmCs) that result from the addition of glucose to the 5 position of cytosine via a glucosyltransferase to form 5-hmC.

Modification of the hmN in a DNA of interest results in a mhmN. For example, transferring a glucose molecule onto a hmN in a target DNA forms a glucosylated hmN (ghmN) such as ghmC. In embodiments of the invention, the hydroxymethylated DNA has a hydroxymethyl group on the C5 position of cytosine. In other embodiments, hydroxymethylation may occur on the N4 position of the cytosine, on the C5 position of thymine or on the N6 position of adenine. The methods described herein are broadly applicable to differentiating any mN or hmN at any position that additionally may be modified as described above. Selective modification of hmN in a DNA may be achieved enzymatically. For example, a sugar molecule such as glucose may be added to an hmN by reacting the DNA with a sugar transferase such as a glucosyltransferase. In the examples, a glucose is added to hmC using recombinant BGT. It was found that AGT works well when used in place of BGT; hence, wherever the use of BGT is described in the text and the examples, it may be substituted by AGT. Moreover, glucosyltransferases from phages T2 and T6 may be substituted for phage T4gt.

The mhmC is subsequently discriminated from mC and C in a cleavage reaction that would not otherwise have discriminated between hmC and mC. An additional example of an enzyme that modifies hmN is a glucosidase isolated from Trypanosomes that glucosylates hydroxymethyluracil (hmU) (Borst et al. Annu Rev Microbiol. 62:235-51 (2008)).

Selective modification of hmC may be achieved chemically, for example, by binding a non-enzyme reagent to an hmC that blocks site-specific endonuclease cleavage, which would otherwise occur. Such chemical reagents may be used exclusively or in conjunction with additional molecules that label the hmC so that DNA containing hmC can be visualized or separated by standard separation techniques from DNA not containing modified hmC. Examples of non-enzyme reagents include antibodies, aptamers, protein labels such as biotin, histidine (His), glutathione-S-transferase (GST), chitin-binding domain or maltose-binding domain, chemiluminescent or fluorescent labels. Alternatively, selective chemical modification of hmC could be employed. This addition could by itself block site-specific endonuclease cleavage, or could bind additional non-enzyme reagents, such as those just described, to either block cleavage, allow visualization, or enable separation.

The modification of hmC results in altered cleavage patterns with a variety of different classes of enzymes. This provides an opportunity for exquisite resolution of individual or clustered hmC in a genome resulting from the varying specificities of the enzymes utilized as well as comprehensive mapping. Additional advantages include visualization of hmN molecules in the DNA of interest using chemical or protein tags, markers or binding moieties.

In an embodiment of the invention, the occurrence of an hmC at a genomic locus can be determined de novo or matched to a predetermined genomic locus using embodiments of the methods described herein for detecting hmC in a nucleic acid molecule or nucleic acid molecule preparation derived from a cell, a tissue or an organism. When used herein, the term “nucleic acid molecule” can be equally used with the term “polynucleotide”.

Embodiments of the methods of the invention may be used to detect an hmC in a nucleic acid molecule so as to compare nucleic acid molecules from a single tissue from a single host or a plurality of nucleic acid molecules from a plurality of tissue samples from a single host with a reference genome or locus, or to compare a plurality of nucleic acid molecules from a single tissue from a plurality of hosts or a plurality of nucleic acid molecules from a plurality of tissues from a plurality of hosts with each other.

In additional embodiments, a method is provided for quantifying the occurrence of an hmC at a genomic locus by analyzing a nucleic acid molecule from a plurality of cells, a tissue or an organism using a quantification method known in the art such as qPCR, end-point PCR, bead-separation and use of labeled tags such as fluorescent tags or biotin-labeled tags.

In an embodiment of the invention, a method is provided for detecting an hmC in a nucleic acid molecule and comparing the occurrence of the hydroxymethylation in a first nucleic acid molecule with the occurrence of an hmC in a second nucleic acid molecule. Another embodiment of the invention, additionally comprises correlating the occurrence of the hmC at an identified locus, which may be predetermined, with a phenotype, i.e., phenotype designation.

A “phenotype designation” refers to a coded description of a physical characteristic of the cell, tissue or organism from which the nucleic acid molecule is derived which is correlated with gene expression and with the presence of an hmC. The phenotype being designated may be, for example, a gene expression product that would not otherwise occur, a change in a quantity of a gene expression product, a cascade effect that involves multiple gene products, a different response of a cell or tissue to a particular environment than might otherwise be expected, or a pathological condition as described herein.

Comparisons of hydroxymethylation patterns throughout the genome and at specific loci provide the basis for a growing database that can provide useful biomarkers for prognosis, diagnosis and monitoring of development, health and disease of an organism.

An “analog” of hydroxymethylcytosine which can be used in the inventions methods alternatively or additionally to hydroxymethylcytosine as such, includes, but is not limited to, labelled hydroxymethylcytosine (e.g. detectably labelled with fluorophores, radioactive tracers, enzyme labels etc.—these detectable labels do preferably not affect the reactions steps which characterize the methods of the present invention) and/or otherwise modified hydroxymethylcytosine (e.g. hydroxymethylcytosine which carries protection groups or other chemical substituents). These analogues are in some embodiments characterized as follows: on the one hand, they can be employed during the synthesizing step (b) of the inventions methods (i.e. the synthesis of the at least one copy of at least a portion of the complementary strand is still possible). On the other hand, they can be used in the reacting step of (c) of the inventions methods, i.e. said modification which characterizes the analogues of hydroxymethylcytosine does not negatively affect the cleavage by said endonuclease of step (c). “Does not negatively affect” means that a cleavage is still possible although it might be that the turnover rate of the respective endonuclease might be decreased due to the presence of the incorporated analog.

It is also envisaged to employ an analog within the context of the methods of the present invention which can be employed during the synthesizing step (b) of the inventions methods (i.e. the synthesis of the at least one copy of at least a portion of the complementary strand is still possible) but which needs a chemical manipulation before it can be used in the reacting step of (c) of the inventions methods. Such modifications are well-known to the technical expert in the field of nucleic acid synthesis and include protection groups or other chemical modifications/substituents which should be removed, cleaved off or replaced before the actual cleavage reaction takes place.

The “product obtained in (b)” is preferably the synthesizing batch of step (b) as such. It is however also envisaged to purify the end product of step (b) of the methods of the invention (which “end product” is the generated double stranded nucleic acid) in order to increase the amount of said double stranded nucleic acid for the subsequent relation step (c) of the inventions methods. Alternatively or additionally, it is also envisaged that said “purification” merely or mainly removes some or all ingredients of the synthesizing reaction of step (b) of the inventions methods (for example unwanted buffer ingredients etc.) which could, otherwise, have an unwanted effect on the subsequent endonuclease cleavage. Methods to purify dsDNA are well-known to the skilled person.

A “portion of the complementary strand of the ss nucleic acid” as referred to in the methods of the present invention includes that a second strand of a nucleic acid molecule is synthesized of a length that is sufficient to provide at least the recognition site for an endonuclease capable of cleaving a ds nucleic acid molecule, wherein cleavage by said endonuclease requires a recognition site that contains hmC on opposite strands. Said portion may by synthesized by any suitable technique to synthesize the complementary strand of a ss nucleic acid molecule or by hybridizing a complementary oligonucleotide to said ss nucleic acid molecule. Said oligonucleotide is preferably of a length that is sufficient to provide at least the recognition site for an endonuclease capable of cleaving a ds nucleic acid molecule, wherein cleavage by said endonuclease requires a recognition site that contains hmC on opposite strands.

“An endonuclease being capable of cleaving said ds nucleic acid molecule, wherein cleavage by said endonuclease requires a recognition site that contains hmC on opposite strands” can preferably be selected from one or more of the following enzymes PvuRts1I, PpeHI, EsaSS310P, EsaRBORFBP, PatTI, Ykrl, EsaNI, SpeAI BbiDI, PfrCORFlI80P, PcoORF314P, BmeDI, AbaSDFI, AbaCI, AbaAI, AbaSI, AbaUMB30RFAP and Asp60RFAP, and catalytically active mutants and derivatives thereof, which are described in WO 2011/091146 (see, for example, Table 1 of WO 2011/091146). A particularly preferred endonuclease is PvuRts1I. However, any of these endonucleases can be applied in the methods of the present invention.

(2) A method of determining or evaluating the hydroxymethylation status within a nucleic acid molecule preparation; comprising:
(a) providing a single-stranded (ss) nucleic acid molecule;
(b) synthesizing at least one copy of at least a portion of the complementary strand (for example, by way of a single round amplification) of said ss nucleic acid molecule thereby generating a double-stranded (ds) nucleic acid molecule, wherein said synthesis is carried out in the presence of hydroxymethylcytosine or analog thereof; and
(c) reacting the product obtained in (b) with an endonuclease being capable of cleaving said ds nucleic acid molecule, wherein cleavage by said endonuclease requires a recognition site that contains hmC on opposite strands; and
(d) analyzing the product obtained in step (c).

“Hydroxymethylation status” as used here and in the claims refers to whether hydroxymethylation is present in a nucleic acid molecule or not. If hydroxymethylation is present, any of the amount and/or location of the hmC can be determined in accordance with the methods and means of the invention. For example, on a molecular level, such correlations can help reveal the function of the target DNA itself, including the impact of the modification on the function of neighboring sequences. Such analysis also can identify biomarkers predictive and diagnostic of normal and altered cellular states

(3) A method of determining or evaluating the hydroxymethylation status of a subject containing a nucleic acid molecule preparation; comprising:
(a) providing a single-stranded (ss) nucleic acid molecule;
(b) synthesizing at least one copy of at least a portion of the complementary strand (for example, by way of a single round amplification) of said ss nucleic acid molecule thereby generating a double-stranded (ds) nucleic acid molecule, wherein said synthesis is carried out in the presence of hydroxymethylcytosine or analog thereof; and
(c) reacting the product obtained in (b) with an endonuclease being capable of cleaving said ds nucleic acid molecule, wherein cleavage by said endonuclease requires a recognition site that contains hmC on opposite strands; and
(d) analyzing the product obtained in step (c).

The term “subject” when used herein includes animals such as mammals, including, but not limited to, primates (e.g., humans), cows, sheep, goats, horses, dogs, cats, rabbits, rats, mice and the like. In preferred embodiments, the subject is a human. The compositions, compounds, uses and methods of the present invention are thus applicable to both human therapy and veterinary applications.

(4) A method of diagnosing a disease in a subject, said disease being characterized by an aberrant hydroxymethylation status; comprising:
(a) providing a sample obtained from said subject, said sample comprising a single-stranded (ss) nucleic acid molecule;
(b) synthesizing at least one copy of at least a portion of the complementary strand (for example, by way of a single round amplification) of said ss nucleic acid molecule thereby generating a double-stranded (ds) nucleic acid molecule, wherein said synthesis is carried out in the presence of hydroxymethylcytosine or analog thereof; and
(c) reacting the product obtained in (b) with an endonuclease being capable of cleaving said ds nucleic acid molecule, wherein cleavage by said endonuclease requires a recognition site that contains hmC on opposite strands; and
(d) analyzing the product obtained in step (c).

A “sample”, as used herein, includes, but is not limited to, any quantity of a substance from a living thing or formerly living thing. Such substances include, but are not limited to, blood, serum, urine, synovial fluid, cells, organs, tissues (e.g., brain or liver), bone marrow, lymph nodes, cerebrospinal fluid, and spleen.

It is contemplated that the use of the methods of the invention for the evaluation of hydroxymethylation is beneficial for the diagnosis of disease and for the evaluation of the efficacy of therapeutic treatments

Detection of hydroxymethylation as an indicator of deregulation of gene expression that gives rise to pathologies such as cancer may be achieved using the methods described herein. It is expected that hydroxymethylation status will provide useful prognostic information for the patient.

It is envisaged that a sample from a subject will be analyzed for a hydroxymethylation status at a single locus or multiple loci to provide detection data in accordance with the methods and means of the invention. Detection data may be quantified and compared with data that is retrieved from a database over a network or at a computer station. The quantified data may be evaluated in view of retrieved data and a medical condition determined. This quantified data may be used to update the database stored at a central location or on the network where the database contains correlations of hydroxymethylation and disease status.

(5) The method of any one of items 1-4, wherein step (d) comprises

(i) sequencing, preferably massive parallel sequencing,

(ii) PCR, preferably qPCR, and/or

(iii) primer extension.

The cleavage fragments from the endonuclease digestion can preferably be ligated to external DNA sequences required for selective amplification and/or subsequent analysis such as sequencing, preferably massive parallel sequencing, PCR, preferably qPCR, and/or primer extension
(6) The method of any one of items 1-5, wherein said nucleic acid molecule is genomic DNA (gDNA) or mitochondrial DNA (mtDNA).

As used herein “genomic DNA” may be a mammalian or other eukaryotic genome or a prokaryotic genome but does not include bacterial virus DNA. The nucleic acid molecule investigated or evaluated in the methods of the invention may include additional defined sequences in the form of double- or single-stranded oligonucleotides hybridized to one or both termini. These oligonucleotides may be synthetic and include adapters or primers or labels. “Genomic DNA” as used here and in the claims preferably refers to a DNA that is isolated from an organism or virus and is naturally occurring.

(7) The method of item 4, wherein said disease is a neurodegenerative disease.

The term “neurodegenerative diseases” are a group of disorders characterized by changes in neuronal function, leading in the majority of cases to loss of neuron function and cell death. Neurodegenerative disorders (diseases) include, but are not limited to, Alzheimer's diseases, Pick's disease, diffuse Lewy Body disease, progressive supranuclear palsy (Steel-Richardson syndrome), multisystem degeneration (Shy-Drager syndrome), motor neuron diseases including amyotrophic lateral sclerosis, degenerative ataxias, cortical basal degeneration, ALS-Parkinson's-Dementia complex of Guam, subacute sclerosing panencephalitis, Huntington's disease, Parkinson's disease, synucleinopathies, primary progressive aphasia, striatonigral degeneration, Machado-Joseph disease/spinocerebellar ataxia type 3, or olivopontocerebellar atrophy.

In particular, epigenetic modifications have been proposed to underlie age-related dysfunction and age-related disorders. In humans 5-hydroxymethylcytosine is generated by the oxidation of 5-methylcytosine (5-mC) by the ten-eleven translocation (TET) family of enzymes. Various studies have shown that 5-hmC is present in high levels in the brain. Its lower affinity to methyl-binding proteins as compared to 5-mC suggests that it might have a different role in the regulation of gene expression, while it is also implicated in the DNA demethylation process. Interestingly, various widely used methods for DNA methylation detection fail to discriminate between 5-hmC and 5-mC, while numerous specific techniques are currently being developed. Recent studies have indicated an increase of 5-hmC with age in the mouse brain as well as an age- and gene-expression-level-related enrichment of 5-hmC in genes implicated in neurodegeneration (van den Hove et al. Curr Alzheimer Res. (2012) Jan. 23, 2012). Thus, these findings suggest that 5-hmC may play an important role in the etiology and course of age-related neurodegenerative disorders.

Szulwach et al. (Nat. Neurosci. (2011) 14(12):1607-1616) have also observed that 5-hmC-mediated epigenetic modification is critical in neurodevelopment and diseases. Accordingly, since the means and methods of the present invention allow, inter alia, the diagnosis of diseases based on the hydroxymethylation status of a subject, said means and methods are particularly suitable for the diagnosis of neurodegenerative disorders as described herein, such as Alzheimer's disease.

(8) The method of item 4, wherein said disease is an age-related disease.

(9) The method of item 8, wherein said age-related disease is selected from the group consisting of cardiovascular disease, cancer, arthritis, cataract, osteoporosis, type 2 diabetes, hypertension. In fact, it was shown by Kudo et al. (Cancer Sci. (2012) doi: 10.1111/j.1349-7006.2012.02213.x) that the loss of 5-hydroxymethylcytosine is accompanied with malignant cellular transformation. Similar results as regards loss of 5-hydroxymethylcytosine were observed by Haffner et al. (Oncotarget (2011) 8:627-637) who report that global 5-hydroxymethylcytosine content is significantly reduced in human cancers. Kraus et al (Int J Cancer. (2012) Jan. 10. doi: 10.1002/ijc.27429) also report that low values of 5-hydroxymethylcytosine (5hmC), are associated with anaplasia in human brain tumours. Accordingly, the claimed method is suitable for the diagnosis of cancer or tumour development, such as brain tumours, since loss of 5-hydroxymethylcytosine or the decrease of the 5-hydroxymethylcytosine content is correlated with cancer or tumour development, such as brain tumours.

(10) The method of any of any one of items 1-9, wherein said endonuclease is an endonuclease of the PvuRts1I family.

The PvuRts1I family, which recognizes ghmC and hmC in DNA, is described in WO 2011/025819, U.S. Provisional Application No. 61/296,630 filed Jan. 20, 2010 and Janosi et al. J. Mol. Biol. 242: 45-61 (1994)) and cleave the DNA at an approximately fixed distance from that base.

(11) The method of any one of items 1-10, further comprising applying the methods disclosed in WO 2011/025819, in particular the methods disclosed in the claims as originally filed or comparing the results obtained in step (d) with the methods disclosed in WO 2011/025819, in particular the methods disclosed in the claims as originally. These methods of WO 2011/025819 are:

1. A method of detecting a hydroxymethylated nucleotide (hmN) in a polynucleotide preparation; comprising:
(a) reacting the polynucleotide preparation, in which an hmN in a polynucleotide preparation is modified, with a site-specific endonuclease, the site-specific endonuclease being capable of cleaving a polynucleotide wherein the specific recognition site contains at least a methylated nucleotide (mN) or hydroxymethylated nucleotide (hmN) but not a modified hmN (mhmN);
(b) detecting an uncleaved polynucleotide in the polynucleotide preparation that would otherwise be cleaved but for a modification of the hmN; so as to detect the hmN in the polynucleotide preparation.
2. A method according to item 1, wherein (b) further comprises detecting a cleaved polynucleotide in the polynucleotide preparation.
3. A method according to item 1 or 2, wherein (a) further comprises ligating an adapter to the polynucleotide preparation for amplifying or sequencing an uncleaved polynucleotide.
4. A method according to any of items 1 through 3, wherein (b) further comprises identifying a genomic locus for the detected hmN.
5. A method according to any of items 1 through 4, wherein the polynucleotide preparation is derived from a cell, tissue or organism and wherein (b) further comprises detecting at a predetermined locus in a genome the hmN in the polynucleotide preparation.
6. A method according to any of items 1 through 5, further comprising determining an amount of the hmN in the predetermined locus in the genome from a cell, a tissue or an organism.
7. A method according to any of items 1 through 6, further comprising comparing the amount of hmN in a first polynucleotide preparation and in a second polynucleotide preparation.
8. A method according to any of items 1 through 7, further comprising correlating a difference in the amount of the hmN at a predetermined locus in a first polynucleotide preparation and in a second polynucleotide preparation with a phenotypic trait.
9. A method according to any of items 1 through 8, wherein (a) further comprises reacting the polynucleotide preparation with a PvuRts1I family endonuclease or a Type IV restriction endonuclease.

(12) The method of any one of items 1-10, further comprising comparing the results obtained in step (d) with a reference sample.

By way of example, if one is interested in detecting hmC in a nucleic acid molecule of interest, he follows the teaching of the present invention and can preferably compare the results obtained in step (d) of the methods of the present invention with a reference sample. For the reference sample, step (b) as described herein is not carried out in the presence of hydroxymethylcytosine or analog thereof. However, second strand synthesis can be carried out in the absence of hydroxymethylcytosine or analog thereof. Following that, step (c) as described herein is carried out with the reference sample. Dependent on the presence or absence of hmC in the upper and/or lower strand, the following results, i.e., digestion by an endonuclease being capable of cleaving ds nucleic acid molecules, wherein cleavage by said endonuclease requires a recognition site that contains hmC on opposite strands, may be possible:
(i) if hmC is present in the upper strand and second strand synthesis is made for the lower strand in the presence of hmC, the sample of interest is cleaved, while the reference sample is not cleaved;
(ii) if hmC is present in the lower strand and second strand synthesis is made for the upper strand in the presence of hmC, the sample of interest is cleaved, while the reference sample is not cleaved;
(iii) if hmC is present in the upper and lower strand and second strand synthesis is made for either the upper or lower strand or for both the upper and lower strand in the presence of hmC, the sample of interest is cleaved, and the reference sample is cleaved, too;
(iv) if hmC is not present in the upper and lower strand and second strand synthesis is made for either the upper or lower strand or for both the upper and lower strand in the presence of hmC, the sample of interest is not cleaved, and the reference sample is not cleaved, either

A “reference sample” includes a “reference nucleic acid molecule” and a “reference genome”. A “reference” nucleic acid molecule as used here refers to a nucleic acid molecule optionally in a database with defined properties that provides a control for the nucleic acid molecule or nucleic acid molecule preparation being evaluated or investigated for hydroxymethylation. A “reference” genome includes a genome and/or hydroxymethylome where the hydroxymethylome is a genome on which an hmC has been mapped. The reference genome may be a species genome or a genome from a single source or single data set or from multiple data sets that have been assigned a reference status.

(13) A kit comprising hmC and an endonuclease of the PvuRts1I family. The kit may also comprise adaptors, primers and nucleotides such G, A, T and/or C. hmC contained in the kit is preferably for the application in the generation of at least a portion of the strand complementary to the ss nucleic acid molecule of interest.

(14). The kit of item 13, wherein said endonuclease of the PvuRts1I family is PvuRts1I. The kit is preferably for performing the methods described herein. Preferably, PvuRts1I is contained in a composition as described herein, e.g., said composition is a solution.

(15) The kit of item 13 or 14 which is a diagnostic kit.

Said kit may further comprise package insert and/or instructions comprising instructions on how to use the endonuclease and the hmC. The term “package insert and/or instructions’ is further used to refer to instructions customarily included in commercial packages of diagnostic products, that contain information about the methods, usage, storage, handling, and/or warnings concerning the use of such diagnostic products. The kits of the present invention may further comprise positive and/or negative controls (e.g. control DNA comprising hmC in one or both strands or control DNA derived from a biological sample which control DNA is already characterized or control DNA having no hmC at all). The kits may further comprise means to remove a sample from a subject.

(16) A composition comprising PvuRts1I and about 10% glycerol. Preferably, said composition does not contain SDS and/or Bromphenolblue (BPB). Alternatively, but also preferred, said composition contains SDS and/or Bromphenolblue (BPB).

Preferably, said composition contains a reaction buffer. A preferred buffer is a Tris buffer such as Tris-HCl, Tris-acetate, Bis-tris-propane HCl, preferably at a concentration of about 10, 20, 30, 40 or 50 mM. The pH of the reaction buffer is preferably between 7.0-8.0, more preferably at a pH of about 7.5, 7.6, 7.7, 7.8 or 7.9.

Said reaction buffer preferably comprises a salt characterized by an anion selected from the group consisting of a sulfate, a phosphate, a chloride, an acetate and a citrate, with a chloride being preferred.

The reaction buffer preferably comprises sodium and/or magnesium as a cation.

Preferably, the salt concentration of the reaction buffer is 50-500 mM. More preferably, the salt concentration in the reaction buffer is such that the ionic strength is equal to or above the ionic strength of about 150 mM NaCl.

A particularly preferred salt contained in the reaction buffer is sodium chloride, preferably at a concentration of about 100-200 mM, more preferably 150 mM.

As an additional salt, the reaction buffer preferably contains magnesium chloride or magnesium acetate, preferably at a concentration of about 1 mM, 2, mM, 3 mM, 4 mM, 5 mM or 10 mM.

The reaction buffer may also preferably contain a reducing agent, such as DTT, preferably at a concentration of about 10 mM, 5 mM or 1 mM.

The composition of the present invention which comprises PvuRts1I and about 10% glycerol has preferably cleavage activity on a nucleic acid molecule, in particular on DNA at the sequence ^{hm}CN₁₁₋₁₂/N₉₋₁₀G, whereby cleavage results in two nucleotides 3′ overhang.

### FIGURE LEGENDS

FIG. 1. Selective restriction of ^{hm}C-containing DNA by PvuRTS1I. (A) Purified PvuRTS1I was resolved on a SDS-polyacrylamide gel and stained with coomassie blue. (B) T4 genomic DNA with the naturally occurring pattern of α- and β-glucosylated ^{hm}C, only β-glucosylated ^{hm}C or non-glucosylated ^{hm}C was incubated without or with decreasing amounts of PvuRTS1I as indicated. (C) Reference PCR fragments of 1139, 800 and 500 bp containing ^{hm}C, ^{m}C and unmodified cytosine at all cytosine residues, respectively, were incubated with or without PvuRTS1I as indicated. (B) and (C) show

FIG. 2. Cleavage site of PvuRts1I. A library of PvuRts1I restriction fragments was generated from a 1139 bp PCR fragment containing only hydroxymethylated cytosine residues and the sequence of 133 restriction fragment ends from randomly chosen clones was determined. (A) Graphical map of the fragment ends. A total of 119 analyzed fragment ends (triangles) matched the consensus sequence ^{hm}CN₁₁₋₁₂/N₉₋₁₀G, which was present at 97 sites (thin vertical lines) in the 1139 bp PCR fragment (thick horizontal line). 53 fragment ends related to the sequence motif ^{hm}CN₁₂/N₁₀G (dark green triangles), 37 to ^{hm}CN₁₁/N₁₀G (bright green triangles) and 14 to ^{hm}CN₁₁/N₉G (light green triangles), while 15 fragment ends matching the consensus sequence ^{hm}CN₁₁₋₁₂/N₉₋₁₀G could not assigned unambiguously to any of these subsets (grey triangles). 14 fragment ends did not match the prevalent consensus sequence (grey circles, see Supplementary FIG. S3). (B) Occurrence of the three subsets of cleavage sites and LOGO representation of the corresponding consensus sequence. The absolute height of each position reflects its overall conservation, while the relative height of nucleotide letters represents their relative frequency. The slash in the three cleavage sequence subtypes indicates the exact cleavage site.

FIG. 3. Differential activity of PvuRts1I on sites with symmetric and asymmetric ^{hm}C. Ninety-four by long substrates with identical sequence were generated that contain a single PvuRts1I consensus site (CN₁₂/N₁₀G) with ^{hm}C or ^{m}C in symmetrical and asymmetrical configurations or no modified cytosine. (A) Strategy for generation of the substrates by PCR amplification in the presence of modified nucleotides. The size of the PvuRts1I digestion products is indicated. (B) The variously modified substrates were digested with the indicated amounts of PvuRts1I and digestion products were resolved on polyacrylamide gels. Note the reduced but tangible digestion of the substrate containing asymmetric ^{hm}C.

FIG. 4. Restriction of mouse genomic DNA by PvuRts1I reflects ^{hm}C content. Genomic DNA from mouse cerebellum or TKO ESCs was mixed with three reference PCR fragments of 1139, 800 and 500 bp containing ^{hm}C, ^{m}C and unmodified cytosine at all cytosine residues, respectively, and incubated with or without PvuRts1I as indicated. Digests were resolved on a 0.8% agarose gel stained with ethidium bromide. Line scans of the gel lanes are aligned to the image of the gel. Red and blue lines correspond to samples incubated with and without enzyme, respectively. Arrows point to the main difference in the profiles form cerebellum and TKO ESC DNA digested with PvuRts1I (red lines).

FIG. 5 (Supplementary FIG. S1). Optimization of PvuRts1I restriction conditions using non-glucosylated T4 genomic DNA as substrate. (A-B) Comparison of cleavage rates in the presence different ionic strength conditions and types and concentrations of bivalent ions. One μg of DNA was digested with 1 U of enzyme in buffer containing 20 mM Tris pH 8.0 and (A) 5 mM MgCl₂ and the indicated concentrations of NaCl or (B) 150 mM NaCl and the indicated concentrations of MgCl₂ or CaCl₂. (C) Combined time course and enzyme titration in buffer containing 20 mM Tris pH 8.0, 150 mM NaCl and 5 mM MgCl₂.

FIG. 6 (Supplementary FIG. S2). Characterization of PvuRts1I activity under different pH (A), detergent conditions (B) and temperature (C). Non-glucosylated T4 genomic DNA was used as substrate. In A and C incubation was for 15 min at 22° C.

FIG. 7 (Supplementary FIG. S3). Cleavage site of PvuRts1I as deduced from a restriction fragment library from the whole non-glucosylated T4 genome. A total of 161 fragment ends were sequenced. 137 fragment ends matched the consensus sequence ^{hm}CN₁₁₋₁₂/N₉₋₁₀G, of which 54 related to the sequence motif ^{hm}CN₁₂N₁₀G, 38 to ^{hm}CN₁₁/N₉₋₁₀G, 15 to ^{hm}CN₁₁/N₉G, while 30 could not be assigned unambiguously to any of these subsets due to the occurrence of multiple ^{hm}C residues upstream of the cleavage site. 24 fragment ends had at least one ^{hm}C residue at a distance 10-13 nucleotides from the cutting site, but no guanine was present in the T4 genomic sequence 10-11 nucleotides downstream the cleavage site. Shown is the occurrence (left) and LOGO graphic representation (right) of the three consensus sequence subtypes. In the graphic representations the absolute height of each position and the relative height of each nucleotide letter reflect overall conservation and relative nucleotide frequency, respectively (Crooks et al., 2004).

FIG. 8 (Supplementary FIG. S4). Sequences form the T4 genomic 1139 bp fragment cut by PvuRts1I that deviate from the predicted consensus sequence ^{hm}C N₁₁₋₁₂/N₉₋₁₀G. All cytosine residues are hydroxymethylated but for simplicity they are here indicated as Cs. ^{hm}C and guanine residues 11-13 nucleotides upstream of and 9-10 nucleotides downstream to the cleavage site, respectively, are highlighted in red. Residues 21-23 nucleotides downstream of a ^{hm}C are shaded in light red.

FIG. 9 (Supplementary FIG. S5). Distribution of the sequenced PvuRts1I restriction fragments over the 1139 bp genomic fragment from T4. The sequences determined form clone inserts are shown in green and aligned to the sequence of the 1139 bp genomic fragment (in black type), while the sequences corresponding to the prevalent PvuRts1I recognition site ^{hm}C N₁₁₋₁₂/N₉₋₁₀ G are shown above the sequence; the sites corresponding to fragments of the library that were actually sequenced are shown in red. The positions corresponding to the two nucleotide 3′ overhangs left by PvuRts1I digestion are highlighted in red and grey for experimentally determined and only predicted sites, respectively. The sequences of the primers used for amplification of the fragment 1139 bp T4 genomic fragment are highlighted in green.

FIG. 10 (Supplemental FIG. S6). Analysis of sequences from the T4 genomic 1139 bp fragment matching the PvuRts1I consensus cleavage site ^{hm}CN₁₁₋₁₂/N₉₋₁₀G that were not found among the sequenced fragments. In the LOGO graphic representations on the right the absolute height of each position and the relative height of each nucleotide letter reflect overall conservation and relative nucleotide frequency, respectively (Crooks et al., 2004).

FIG. 11 (Supplementary FIG. S7). Confirmation of a two nucleotide 3′ overhang cleavage pattern by PvuRts1I. A 140 bp fragment containing only hydroxymethylated cytosine residues and a single PvuRts1I site was amplified from the T4 genome and digested with PvuRts1I. The two ensuing PvuRts1I restriction fragments were directly sequenced from their respective 5′ ends employing the same primers used for amplifying the original 140 bp fragment. Alignment of the two sequence tracks to the original sequence revealed a two nucleotide gap consistent with a 3′ overhang configuration of these nucleotides at PvuRts1I ends. Only the ends of the sequence tracks corresponding to the PvuRts1I site are shown. The appropriately spaced ^{hm}C residues on either side of the cleavage site and opposite strands that constitute the PvuRts1I site are highlighted. The large adenine peaks (green) present at the end of each sequence track but not in the original sequence are due to addition of a 3′ overhanging adenine residue by the DNA polymerase used for the sequencing reaction.

FIG. 12 (Supplementary FIG. S8). Identification of PvuRts1I fragments from substrates with increasing ^{hm}C content. (A) The proximal upstream regulatory region of the nanog locus (region III) was amplified in the presence of increasing concentrations of 5-hydroxymethyl-dCTP, yielding fragments with randomly distributed ^{hm}C sites in the respective proportions (not shown). These fragments were digested with PvuRts1I and ligated to linkers with random two nucleotide overhangs to match PvuRts1I ends. Ligation products were amplified with two distinct nanog specific primers (nanog P1 and P2) each paired with a linker specific primer. The PCR products obtained are shown in (B). The percentage of hmC in the original substrate fragments and the presence of the linker in the ligation reaction are indicated. NTC: no template control. (C) Products from PCR reactions shown in (B) were randomly cloned and sequenced. The numbers of sequences containing ends corresponding to the PvuRts1I consensus and site subtype are reported. The asterisk demarks a sequence that could not be univocally assigned to ^{hm}CN₁₂/N₉G or ^{hm}CN₁₁/N₉G due to the presence of consecutive C residues and is reported under both categories. In the case of substrates containing 10% ^{hm}C both primer sets yielded fragments with specific PvuRts1I digestion products that mapped to several predicted cleavage sites (not shown). We note that 1% ^{hm}C is in the same range as measured only in mouse tissues with the highest global ^{hm}C content (3,4,6-9,23). It follows that high local concentrations of ^{hm}C sites facilitate detection by PvuRts1I with this procedure.

FIG. 13. 275 bp DNA fragment from the human nanog promoter (SEQ ID NO: 1). Positions are relative to the ATG of nanog. PvuRts1I recognition sites (^{hm}C N₁₁₋₁₂/N₉₋₁₀ G) are shown above the sequence with the central stars indicating the position of two nucleotide 3′ overhangs left by PvuRts1I digestion. The recognition site used for the detection experiment is marked in red (between position −2067 and −2044). The primers used for amplification of the fragment and for ^{hm}C detection are highlighted in yellow (Nanog-FWD, Detection primer, Nanog-REV short). Positions are relative to the ATG of nanog.

FIG. 14. Quality control of 275 bp DNA substrates with different hmC contents. 50-100 ng PCR fragments per lane were separated on a 1.5% TAE agarose gel at 8 V/cm for 20 min. 100 bp Ladder (New England Biolabs) was used as size standard.

FIG. 15. Test digestion of 275 bp DNA substrates with hmC contents of 0% and 100%. Digestion products were separated on a 1.5% TAE agarose gel at 8 V/cm for 20 min. 100 bp Ladder (New England Biolabs) was used as size standard.

FIG. 16. PvuRts1I digestion of substrates. Substrates used for digestion and digestion products (50 ng each) were separated on a 1.5% TAE agarose gel at 8 V/cm for 20 min. 100 bp Ladder (New England Biolabs) was used as size standard. Please note the difference in the amount of digestion fragments obtained between samples “10% hmC” and “10% hmC 2ss”. 2nd s. s., second strand synthesis.

FIG. 17. Sequence of the 71 bp ^{hm}C detection product (SEQ ID No: 6). To selectively detect fragments cut by PvuRts1I at the position indicated in red, for real time PCR of the ligated products the linker specific primer M13(-20) and the nanog specific Detection primer were used. Primer sequences (Detection primer, M13(-20)-REV, AT adapter) are highlighted in yellow. Position is relative to the ATG of nanog.

FIG. 18. Quality control of real time PCR. Amplification products were separated on a 2% TAE agarose gel at 8 V/cm for 15 min. 100 bp Ladder (New England Biolabs) was used as size standard. Please note the appearance of unspecific amplification products especially in samples “0% ^{hm}C”, “0.1% ^{hm}C”, and “1% ^{hm}C”. 2nd s. s., second strand synthesis.

FIG. 19. Quantification of ligation products. Values are the mean from 4 technical replicates and normalized to 100% ^{hm}C. The upper graph shows the result with 2^{nd strand synthesis, while the lower graph shows the result without} 2^{nd} strand synthesis. Error bars indicate standard deviation.

### EXAMPLES

### Materials and Methods

Cloning and Purification of PvuRts1I

The sequence encoding PvuRts1I was synthesized at Mr. Gene GmbH (Regensburg) and cloned into the pET28a vector (Novagen). BL21(DE3) E. coli cells carrying the expression vector were grown in LB medium at 37° C. until A₆₀₀=0.6-0.7 and induced with 1 mM isopropyl β-d-thiogalactopyranoside for 16 h at 18° C. Lysates were prepared by sonication in 300 mM NaCl, 50 mM Na₂HPO₄ pH 8.0, 10 mM imidazole, 10% glycerol, 1 mM β-mercaptoethanol), cleared by centrifugation and applied to a nickel-nitrilotriacetic acid column (QIAGEN) pre-equilibrated with lysis buffer. Washing and elution were performed with lysis buffer containing 20 and 250 mM imidazole, respectively. Eluted proteins were applied to a Superdex S-200 preparative gel filtration column (GE Healthcare) in 150 mM NaCl, 20 mM Tris, pH 8.0, 10% glycerol, 1 mM DTT and peak fractions were pooled. The stability of PvuRts1I upon storage was improved by supplementation with 10% glycerol.

Preparation of DNA Substrates

In vivo α/β-glucosylated and non-glucosylated T4 phage DNA was isolated essentially as described (4). Briefly, T4 stocks were propagated on E. coli strain CR63, which was also used for the isolation of glucosylated T4 DNA. To isolate non-glucosylated T4 DNA, wild type T4 phage was amplified on a ER1565 ga/U mutant strain. β-glucosylated T4 DNA was generated in vitro by treatment of non-glucosylated T4 DNA with purified T4 β-glucosyltransferase (7). Genomic DNA was isolated from mouse cerebellum and TKO ESCs (21) as described (7).

Reference DNA fragments containing exclusively ^{hm}C, ^{m}C or unmodified cytosine residues were prepared by PCR using 5-hydroxymethyl-dCTP (Bioline GmbH), 5-methyl-dCTP (Jena Bioscience GmbH) and dCTP, respectively. T4 phage DNA template, Phusion HF DNA Polymerase (Finnzymes) and primer 5′-GTG AAG TAA GTA ATA AAT GGA TTG-3′ (SEQ ID NO: 9), which does not contain cytosine residues, were used for amplification of all reference DNA fragments. To generate the reference 1139 bp fragment with 100% ^{hm}C for restriction with PvuRts1I the second primer was 5′-TGG AGA AGG AGA ATG AAG AAT AAT-3′ (SEQ ID NO: 10), which also does not contain cytosine residues. To generate the 800 and 500 bp control substrates containing only ^{m}C and only unmodified cytosine for restriction with PvuRTS1I the second primer was 5′-GCC ATA TTG ATA ATG AAA TTA AAT GTA-3′ (SEQ ID NO: 11) and 5′-TCA GCA ATT TTA ATA TTT CCA TCT TC-3′ (SEQ ID NO: 12), respectively. PCR products were purified by gel electrophoresis followed by silica column purification (Nucleospin, Macherey-Nagel). The 140 bp fragment used to determine the orientation of the PvuRTS1I cleavage overhang was amplified with primers 5′-TAT ACT GAA GTA CTT CAT CA-3′ (SEQ ID NO: 13) and 5′-CTT TGC GTG ATT TAT ATG TA-3′ (SEQ ID NO: 14).

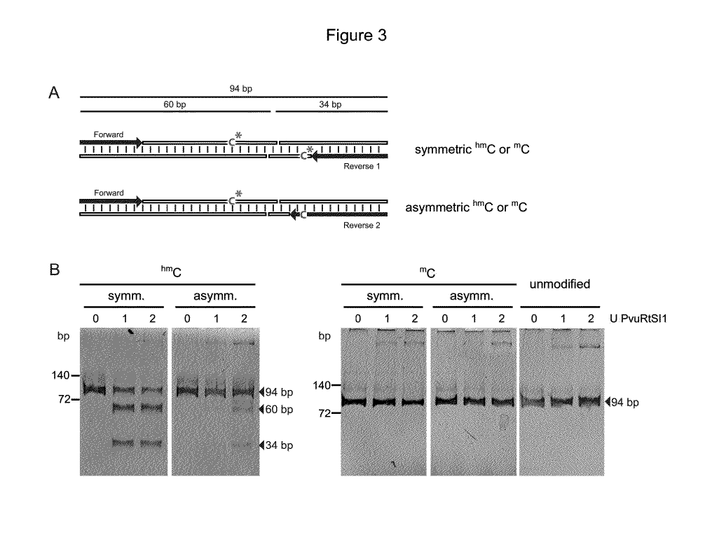

For the preparation of substrates with a single PvuRts1I consensus containing hmC or mC in symmetrical or asymmetrical configuration a 94 bp fragment was amplified from the T4 genome with primers 5′-CTC GTA GAC TGC GTA CCA ATC TAA CTC AGG ATA GTT GAT-3′ (SEQ ID NO: 15) and 5′-TAT GAT AAG TAT GTA GGT TAT T-3′ (SEQ ID NO: 16). This fragment contains a single site corresponding to the identified PvuRts1I consensus hmCN11-12/N9-10G (SEQ ID NO: 27) and was used as a template according to the strategy depicted in FIG. 3. To generate substrates with symmetric cytosine modifications or unmodified cytosine the fragment was amplified with forward primer 5′-CTC GTA GAC TGC GTA CCA-3′ (SEQ ID NO: 17) and reverse primer 1 5′-TAT GAT AAG TAT GTA GGT TAT T-3′ (SEQ ID NO: 26) in the presence of the respective modified or unmodified dCTP. To generate substrates with asymmetric cytosine modifications the same forward primer was paired with reverse primer 2 5′-TAT GAT AAG TAT GTA GGT TAT TCA A-3′ (SEQ ID NO: 18).

DNA Restriction with PvuRts1I and Identification of Cleavage and Recognition Site

Unless otherwise stated the reaction conditions contained 150 mM NaCl, 20 mM Tris, pH 8.0, 5 mM MgCl₂, 1 mM DTT. One unit of PvuRTS1I was defined as amount of enzyme required to digest 1 μg of ^{hm}C-containing T4 DNA in 15 min at 22° C. For assessment of enzyme specificity 100 ng of each control fragment were digested separately or together with 200 ng of genomic DNA in 30 μl reactions containing standard buffer and 1 U of purified PvuRts1I at 22° C. for 15 min.

For identification of the cleavage and recognition site the 1139 bp, fully hydroxymethylated fragment amplified from the T4 genome or whole non-glucosylated T4 DNA were digested under standard conditions. Fragment ends were blunted with Klenow polymerase (NEB) and cloned using the Zero Blunt® PCR Cloning Kit (Invitrogen). Randomly selected clones were sequenced and the data were analyzed using WebLogo (22).

### Supplementary Methods

Generation of Fragments from the Nanog Upstream Region III Containing Known Levels of ^{Hm}C, PvuRts1I Digestion and Identification of Digestion Products.

Genomic DNA from JM8A3.N1 ESCs (EUCOMM, Helmholtz Center Munich, Neuherberg, Germany) was isolated using the NucleoSpin Triprep Kit (Macherey-Nagel). To prepare substrates containing different hmC levels (0%, 1%, 2.5%, 5%, 10%), genomic DNA from JM8A3.N1 cells was used as a template to amplify a 867 bp fragment from region III of the nanog promoter (Hattori et al, Genes to cell, 2007) using corresponding ratios of 5-hydroxymethyl-dCTP (Bioline GmbH) and dCTP, Phusion HF DNA Polymerase (Finnzymes) and the following primers: nanog for 5″-TCA GGA GTT TGG GAC CAG CTA-3″ (SEQ ID NO: 19) and nanog rev 5″-CCC CCC TCA AGC CTC CTA-3″ (SEQ ID NO: 20). After purification of the PCR fragments using the NucleoSpin Extract II kit (Macherey-Nagel), 250 ng of each fragment was digested with 2 U of PvuRTS1I for 15 min at 22° C. and the enzyme was heat inactivated at 60° C. for 20 min. Twentyfive nanograms of digested fragment were ligated to a linker containing random two nucleotide 3″ overhangs, generated by annealing the following primers: For 5″-CTC GTA GAC TGC GTA CCA TG NN-3″ (SEQ ID NO: 21) and Rev 5″-CA TGG TAC GCA GTC TAC CAG-3″ (SEQ ID NO: 22). The ligation reaction was carried out using T4 DNA Ligase (NEB) overnight at 16° C. As a control for ligation specificity, each fragment was ligated in the absence of the linker. To selectively amplify fragments cut by PvuRTS1I, the ligated products were amplified by PCR with Phusion HF DNA Polymerase (Finnzymes) using a linker specific forward primer (For 5″-CTC GTA GAC TGC GTA CCA TG-3″) (SEQ ID NO: 23) and nanog specific reverse primers (P2: 5″-GAG TCA GAC CTT GCT GCC AAA-3″ (SEQ ID NO: 24) and P1: 5″-GCC GTC TAA GCA ATG GAA GAA-3″) (SEQ ID NO: 25). Libraries of digested and ligated fragments containing 1 and 10% hmC were generated using the Zero Blunt® PCR Cloning Kit (Invitrogen). Randomly selected clones were sequenced and analyzed for the presence of PvuRts1I ends.

### Results

^{hm}C-Specific Endonuclease Activity of PvuRts1I

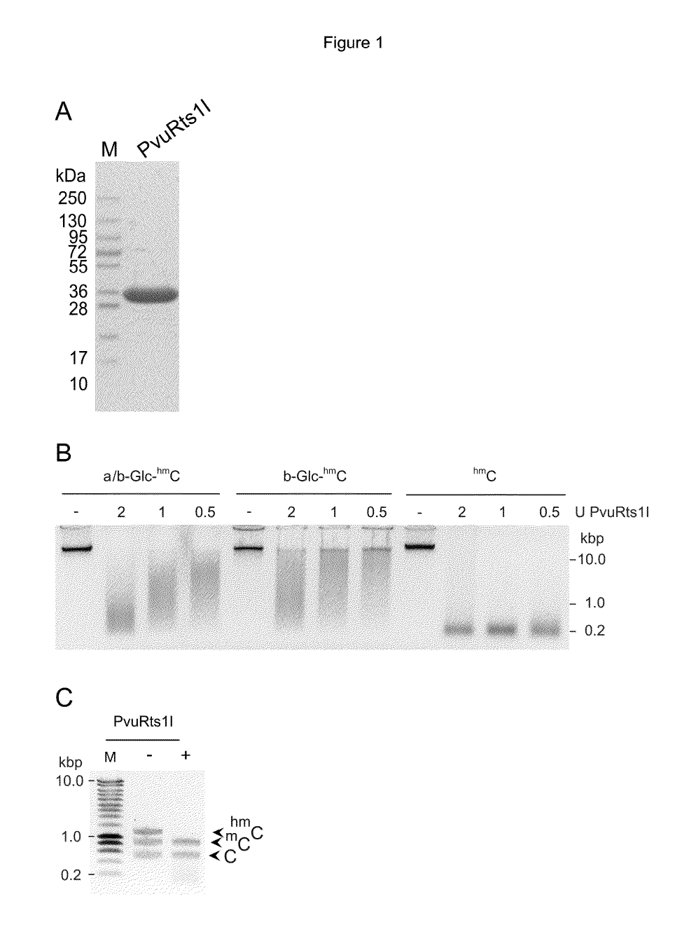

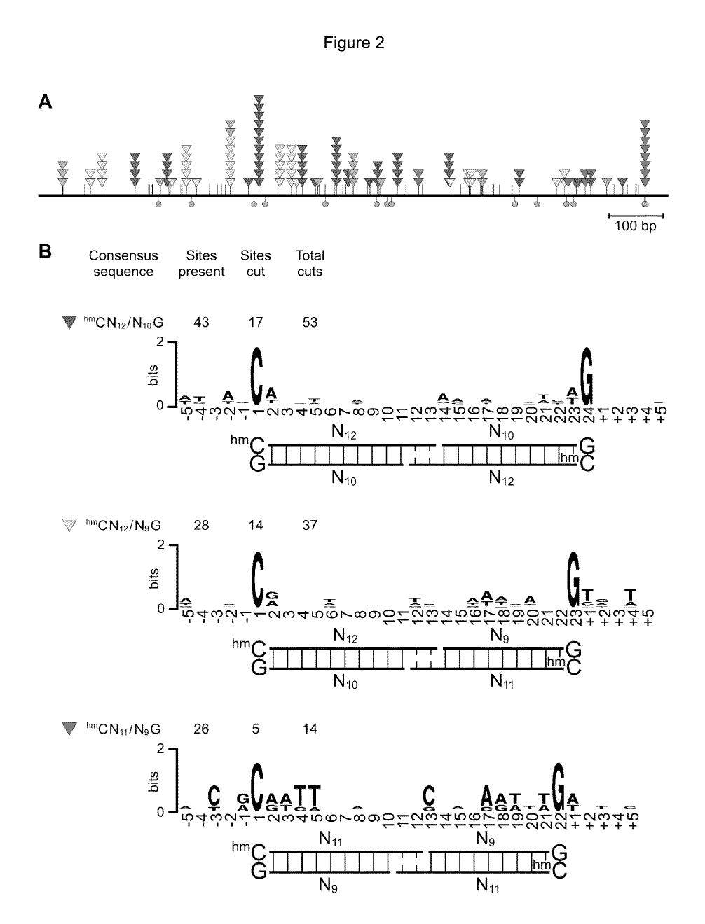

His-tagged PvuRts1I was expressed in E. coli and purified to homogeneity by sequential Ni²⁺ affinity and size exclusion chromatography (FIG. 1A). As bacteria carrying the Rts1 plasmid were shown to restrict the ^{hm}C-containing T-even phages, but not ^{m}c-containing T-odd phages or λ phage, which does not contain modified cytosine (20), we initially used T4 genomic DNA as a substrate to test the activity of purified PvuRts1I. T4 genomic DNA was isolated from both galU⁺ and galU⁻ strains, the latter being UDP-glucose deficient and thus containing only non-glucosylated ^{hm}C. Under the same digestion conditions non-glucosylated T4 DNA was digested more efficiently than both naturally α- and β-glucosylated and in vitro β-glucosylated counterparts (FIG. 1B). Non-glucosylated T4 DNA was cleaved into fragments with an apparent size of about 200 bp, indicating that PvuRts1I recognizes a frequently occurring sequence (FIG. 1B and Supplementary FIGS. S1 and S2). We then used non-glucosylated T4 DNA to test the activity of the enzyme under various conditions. PvuRts1I was strictly dependent on Mg²⁺ ions, which could not be substituted with Ca²⁺, and endonuclease activity was maximal in the presence of 100-200 mM NaCl (Supplementary FIG. SIA and B). However, during purification we observed that the enzyme is unstable in solutions of ionic strength lower than 150 mM NaCl. The activity of PvuRts1I was found highest at pH 7.5-8.0 and was unaffected by the presence of Tween 20 or TritonX-100 (Supplementary FIGS. S2A and B). We also observed that after prolonged incubation PvuRts1I precipitates even at room temperature, consistent with the reported temperature sensitivity of the phage restriction activity in cells carrying the Rts1 plasmid (20). Upon short incubation times maximal activity was observed at 22° C. (Supplementary FIG. 2C). Thus, the relative amounts of enzyme and DNA substrate were standardized so that digestion was complete in 15 minutes at 22° C. in the presence of 150 mM NaCl (Supplementary FIGS. S1C and S2C).

The specificity of PvuRts1I with respect to cytosine modification was further tested by digesting reference fragments containing exclusively unmodified cytosine (500 bp), ^{m}C (800 bp) or ^{hm}C (1139 bp; FIG. 1C). Under standard digestion conditions purified PvuRts1I selectively cleaved the ^{hm}C-containing fragment, consistent with the relative restriction efficiency of bacteriophages with distinct cytosine modifications by bacteria carrying the Rts1 plasmid

Determination of PvuRts1I Cleavage Sites

To identify the cleavage pattern of PvuRts1I we generated libraries of restriction fragments from either the whole T4 genome (Supplementary FIG. S3) or a 1139 bp fragment amplified from the same genome containing exclusively hydroxymethylated cytosines (FIG. 2). Random sequencing of 161 and 133 fragment ends from the whole T4 genome and 1139 bp fragment libraries revealed that 85 and 89%, respectively, matched the consensus sequence ^{hm}CN₁₋₁₂/N₉₋₁₀G. Among these 78 and 87%, respectively, showed one of three similar sequence patterns, ^{hm}CN₁₂/N₁₀G, ^{hm}CN₁₂/N₉G and ^{hm}CN₁₁/N₉G, while for the remaining fragment ends the exact number of nucleotides between the modified cytosine and the cleavage site could not be determined unambiguously due to the occurrence of multiple ^{hm}C residues upstream of the cleavage site. Of the sequenced fragment ends 14 and 11% from the whole T4 genome and 1139 bp fragment libraries, respectively, did not match the ^{hm}CN₁₁₋₁₂/N₉₋₁₀G consensus. However, 100 and 80% of these ends, respectively, contained at least one ^{hm}C residue 10-13 nucleotides upstream of the cleavage site, while no guanine was present in the T4 genomic sequence 10-11 nucleotides downstream the cleavage site (Supplementary FIG. S4). The sequenced clones from the 1139 bp T4 genomic fragment library corresponded to an 81% coverage of the fragment, with some PvuRts1I fragments occurring multiple times, while other fragments that were predicted on the basis of the ^{hm}CN₁₁₋₁₂/N₉₋₁₀G consensus were not found (FIG. 2 and Supplementary FIG. S5). Examination of the missing fragments did not show any common sequence feature beyond the ^{hm}CN₁₁₋₁₂/N₉₋₁₀G consensus (Supplementary FIG. S6), suggesting that their absence from the sequenced fragments was due to limited sampling. Alignment of sequenced fragment ends from the T4 genomic fragment library showed that 2 nucleotides around the cleavage site were missing from all clones, suggesting a 2-nucleotide 3′ overhang cleavage pattern (Supplementary FIG. S5). This was confirmed by direct sequencing of the two fragments generated by digestion of a 140 bp amplicon containing a single PvuRts1I site (Supplementary FIG. S7).

The results above reveal a symmetric nature of the preferred cleavage sites and raise the issue of PvuRTs1I activity on sites with modified cytosine in symmetric and asymmetric configuration. To clarify this issue we used a PCR strategy to generate DNA substrates with identical sequence and containing a single PvuRts1I consensus site with ^{hm}C or ^{m}C in symmetrical and asymmetrical configurations or no modified cytosine (FIG. 3A). In the presence of enzyme amounts that did not cleave substrates with unmodified and ^{m}C sites digestion of substrates with asymmetric ^{hm}C at the PvuRTs1I site was reduced with respect to substrates with symmetric ^{hm}C, but still appreciable.

Residual undigested substrate with symmetric ^{hm}C at the PvuRTs1I site in these reaction conditions was typically observed with such short substrates, but not with longer ones.

Digestion of Mammalian Genomic DNA with PvuRts1I

To investigate cleavage site preference and efficiency of PvuRts1I digestion for mammalian genomic DNA we initially selected the upstream regulatory region III of the mouse nanog gene (23). As this region was shown to be bound by Tet1 and to acquire CpG methylation upon knockdown of Tet1 in embryonic stem cells (ESCs) (5), it represents a potential candidate as a mammalian genomic sequence containing ^{hm}C. Real time amplification of this region from ESC genomic DNA did not show a significant decrease of product after PvuRts1I digestion (data not shown). We then devised a strategy to positively identify rare PvuRts1I digestion products. After PvuRts1I digestion genomic fragments were ligated to a linker with a random two-nucleotide 3′ overhang. Ligation products where then amplified using nanog specific primers paired with a linker specific primer, but no amplification product could be obtained (data not shown). This result may be explained by an extremely seldom occurrence of ^{hm}C at cleavage sites of this locus (especially in symmetric configuration), inefficiency of PvuRts1I digestion or both. In this regard it is important to consider that positive identification of ^{hm}C sites in this region of the nanog locus has actually not been reported for ESCs. In addition, during the revision of the present work a manuscript was published (24) that could not confirm the reduced nanog expression and ESC differentiation previously reported upon Tet1 knockdown (5), raising uncertainty about the actual occurrence of ^{hm}C at the nanog promoter in ESCs.

As there are no clear and quantitative data on the levels and density of ^{hm}C at specific genomic sites available yet we generated defined substrates to validate the PvuRst1I cut-ligation amplification protocol for the identification of ^{hm}C sites. We PCR amplified region III of the nanog promoter in the presence of increasing concentrations of 5-hydroxymethyl-dCTP and confirmed the incorporation of proportional levels of ^{hm}C using the recently reported β-glucosylation assay (7) (data not shown). Fragment samples with increasing ^{hm}C content were then digested with PvuRts1I and the same ligation/PCR strategy for the identification of digestion products was applied as described above (Supplementary FIG. S8A). Detection of fragments with ends corresponding to the PvuRts1I cleavage pattern raised with increasing ^{hm}C content.

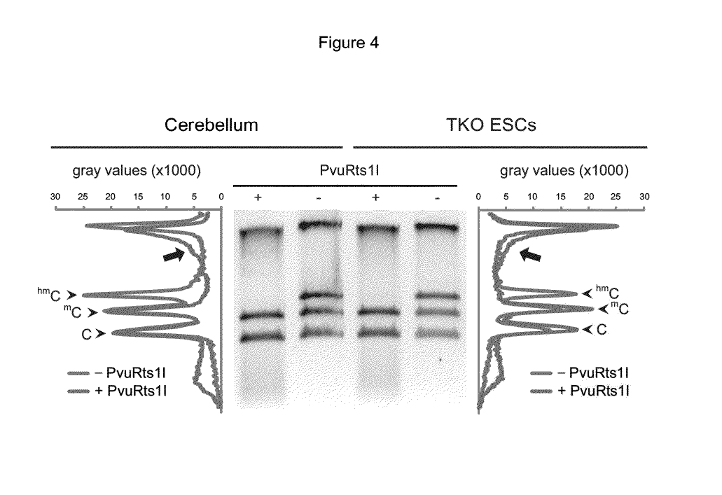

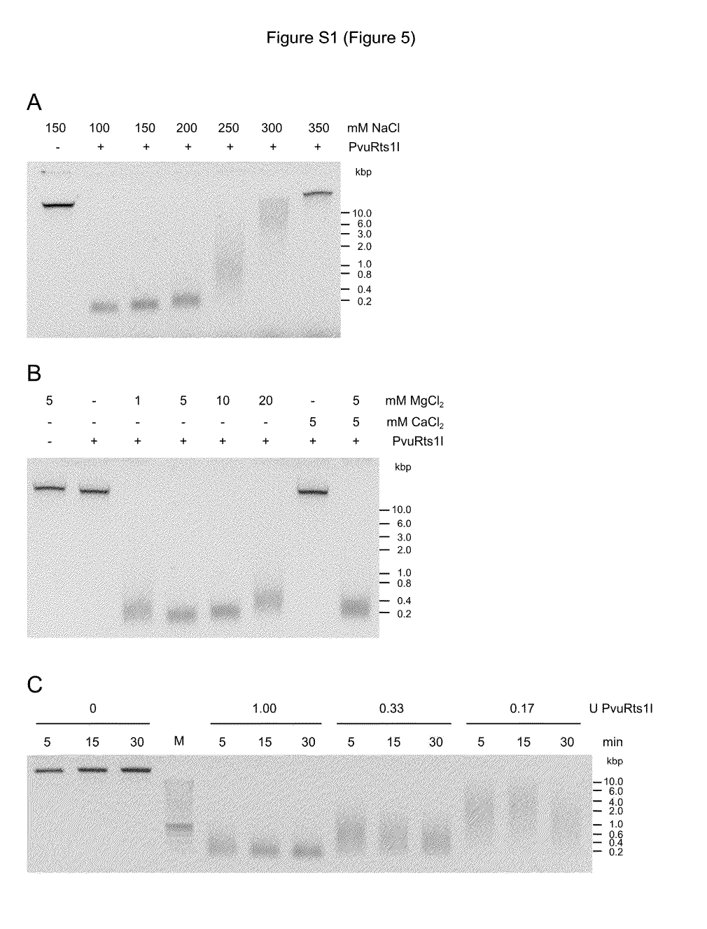

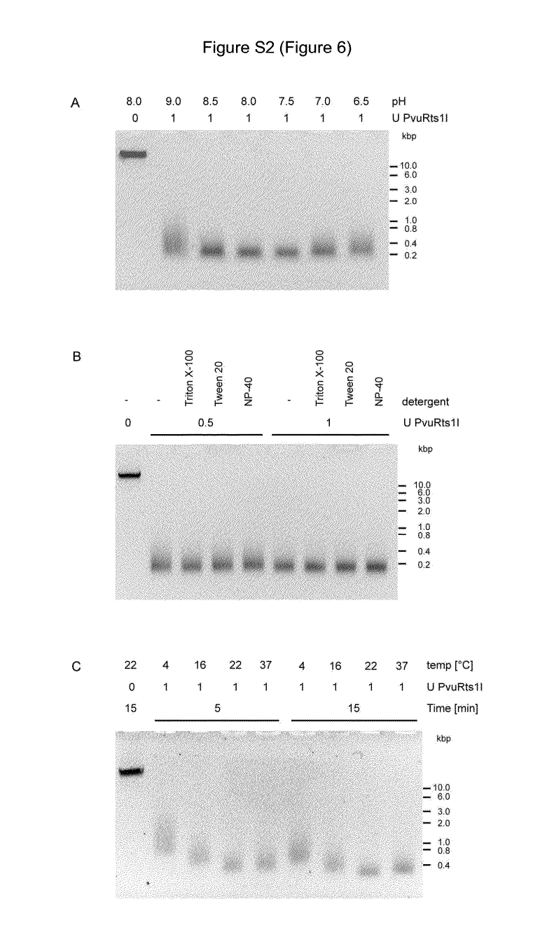

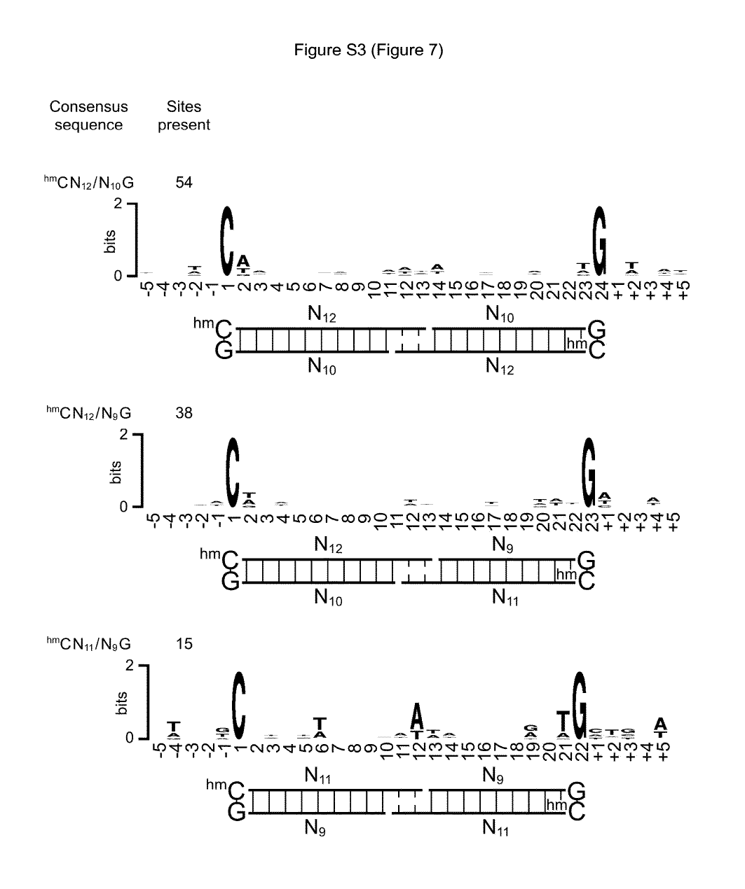

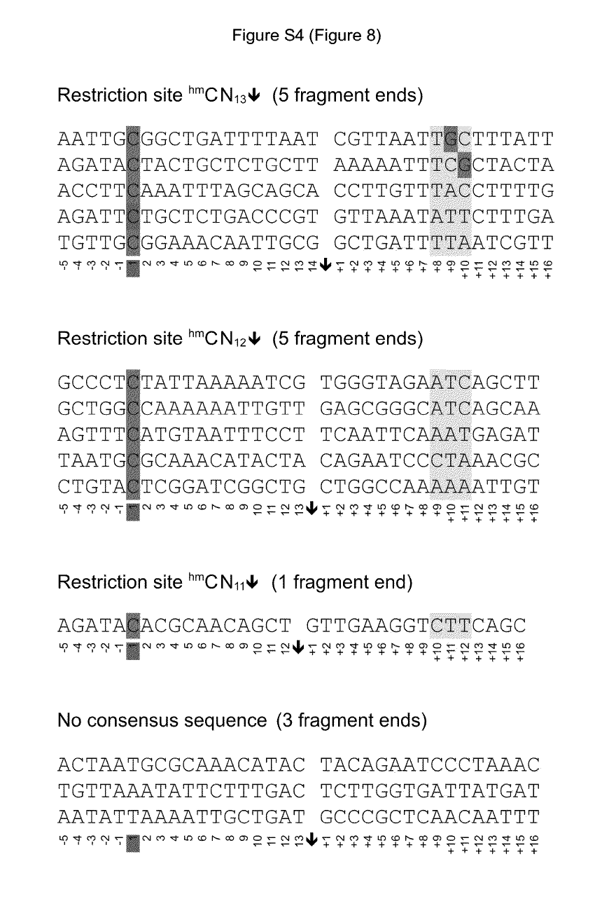

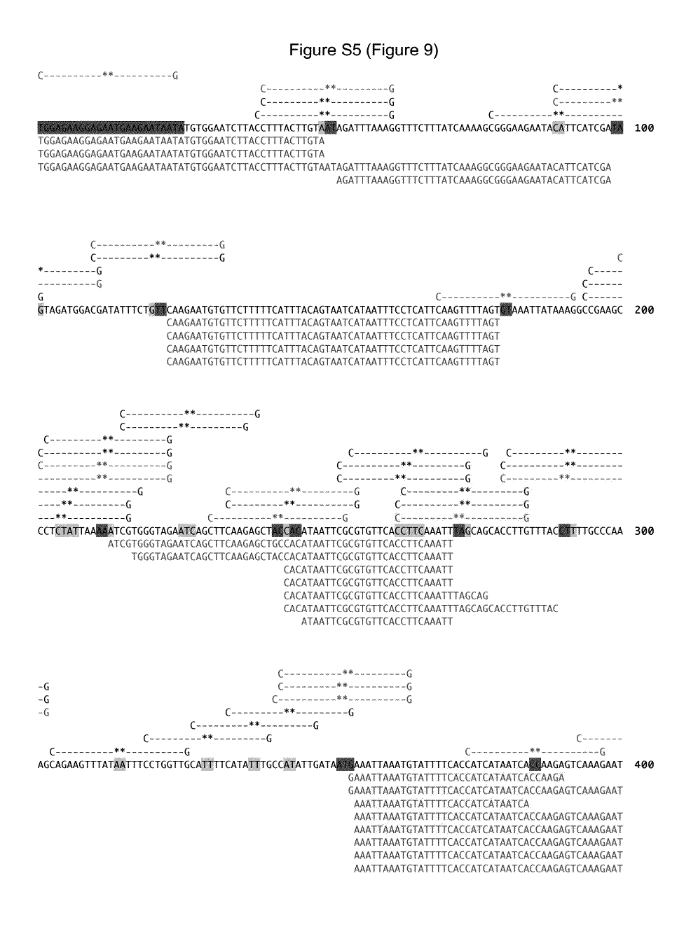

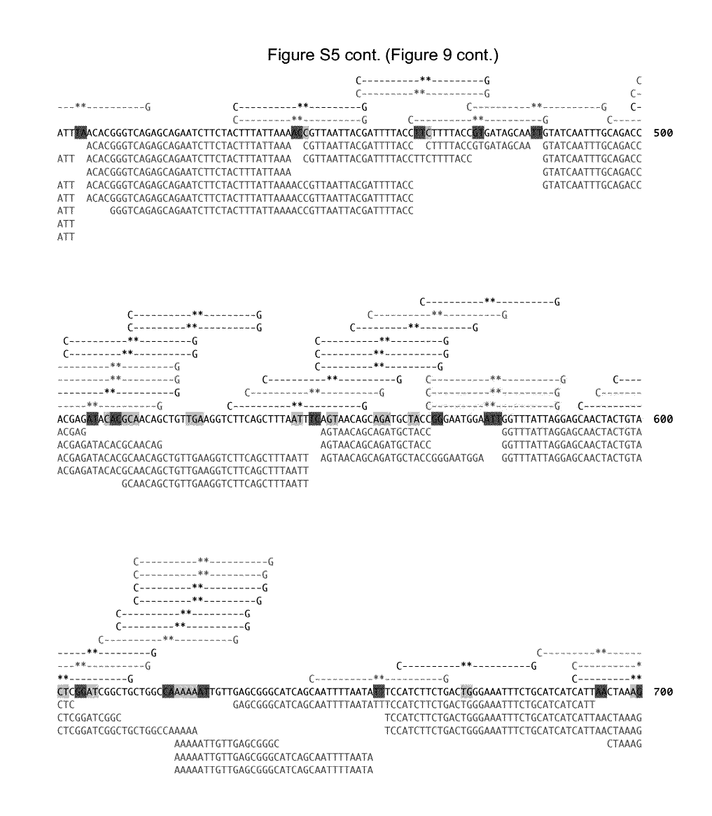

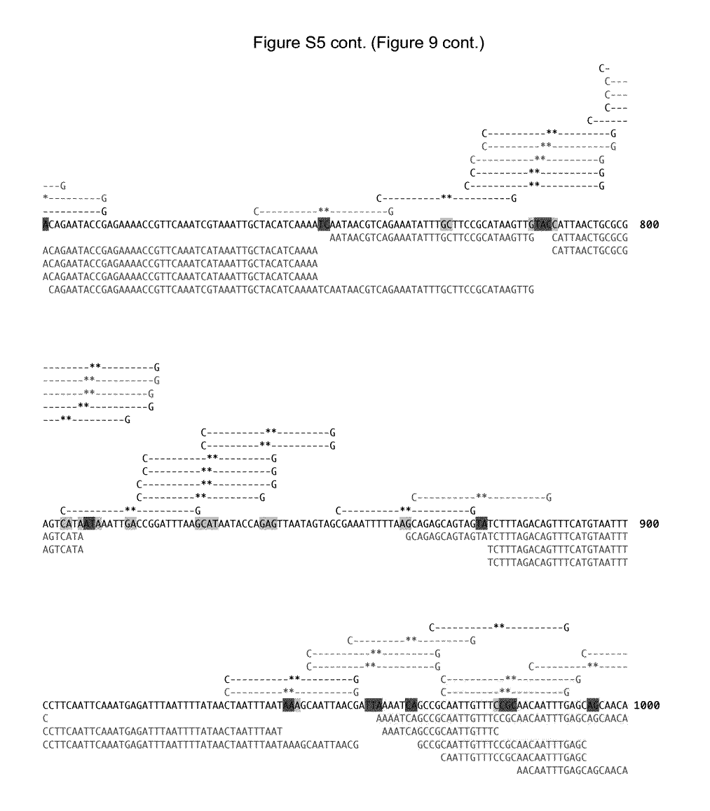

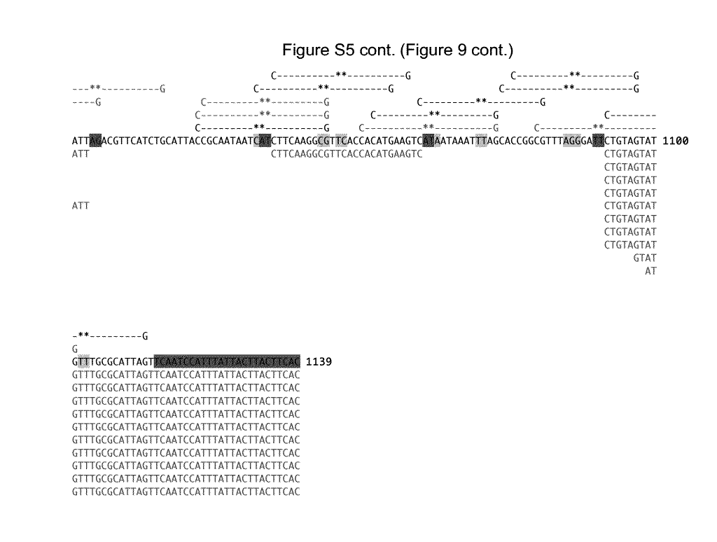

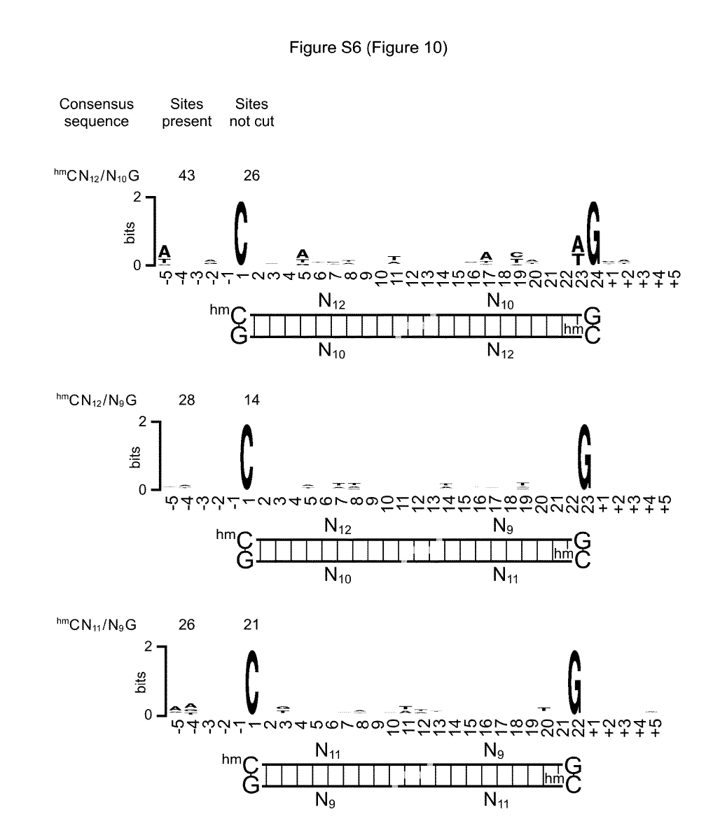

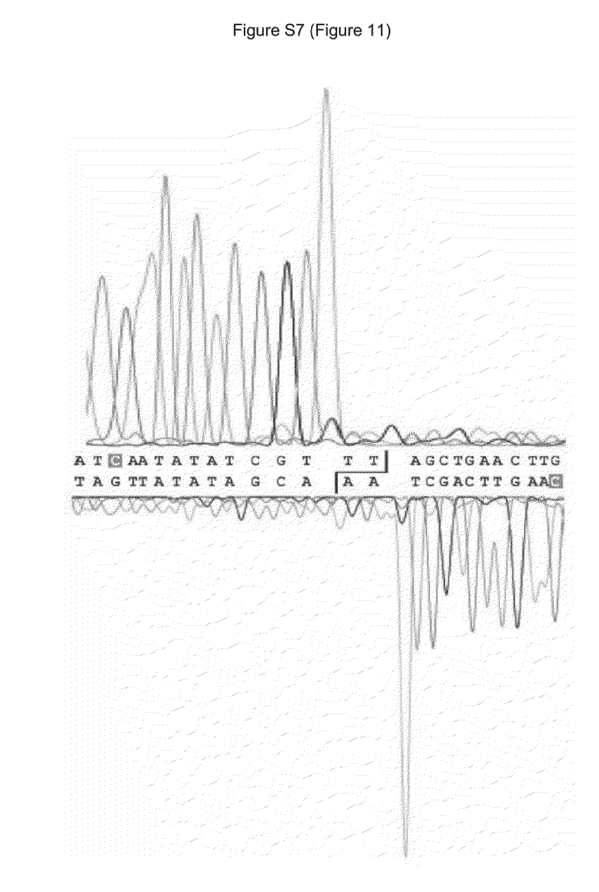

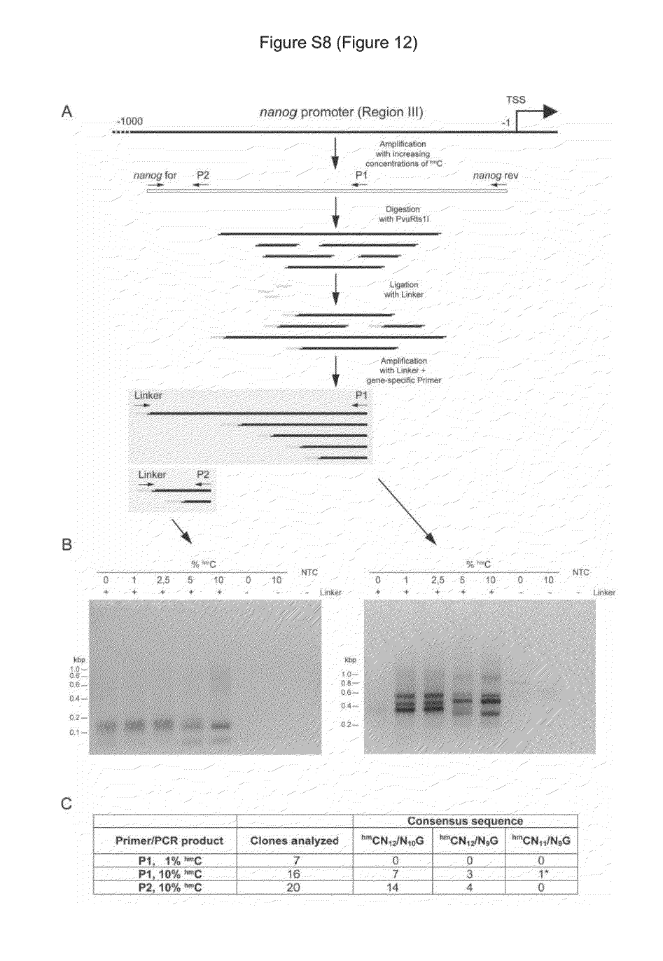

We previously quantified global ^{hm}C levels in genomic DNA from ESCs and adult somatic tissues using in vitro ^{hm}C glucosylation (7). Consistent with other studies (3,6,8,9), this analysis revealed that genomic DNA from adult brain regions has a high ^{hm}C content. In addition, we showed that in ESCs that are triple knockout (TKO) for all three major DNA methyltransferases Dnmt1, 3a and 3b (21) genomic ^{hm}C levels were around the estimated limit of detection, although reproducibly above background. Therefore, we compared the PvuRts1I restriction pattern of genomic DNA from cerebellum and TKO ESCs as representative of samples with high and very low ^{hm}C levels, respectively. As internal controls we co-digested each of the two genomic DNA samples with the same reference fragments as used to test the specificity of PvuRts1I with respect to cytosine modification (FIG. 1C). As expected from the relative low abundance of ^{hm}C in mammalian genomic DNA, there was a limited reduction of high molecular weight fragments and appearance of lower molecular weight smear (FIG. 4). However, DNA from cerebellum was clearly digested to a higher extent than DNA from TKO ESCs as evident from the line scans across the respective gel lanes (FIG. 4). The low but appreciable degree of digestion observed for genomic DNA from TKO ESCs does not seem to result from relaxed specificity or contaminating nuclease activities, as only control substrates containing ^{hm}C, but not ^{m}C or unmodified cytosine, were digested when incubated either separately or together with genomic DNA (FIG. 1C and FIG. 4). Absence of digestion of control substrates containing ^{m}C and unmodified cytosine was evident from the unaltered ratio of their respective signals in the presence and absence of enzyme. This result shows that the extent of digestion by PvuRts1I reflects the relative ^{hm}C content in mammalian genomic DNA.

Detection of 5-Hydroxymethylcytosine (^{hm}C) Residues Via DNA Restriction With PvuRts1I Endonuclease Following Second Strand Synthesis with ^{hm}C

1. Generation of DNA Fragments with Defined ^{hm}C Contents

A 275 bp fragment from the human nanog promoter (position −2272 to −1992 relative to the ATG of nanog) was chosen as substrate for all following steps (FIG. 13; SEQ ID NO: 1). Substrates with different ^{hm}C contents (0%, 0.1%, 1%, 10%, 100%) were prepared using corresponding ratios of 5-hydroxymethyl-dCTP and dCTP, and the following primers: Nanog-FWD (5′-CTC CTG TCT CAG CCT CCC TA-3′) (SEQ ID NO: 2) and Nanog-REV short (5′-AGT TGA GGT TTA GGA AGC TAT CTG-3′) (SEQ ID NO:3).

Amplification was performed in a total volume of 50 μl 1× Phusion HF Buffer (Finnzymes) with 100 ng human genomic DNA (from an ALL cell line) as template, 200 μM each of dATP, dTTP, dGTP, and d^{hm}CTP/dCTP mixes (d^{hm}CTP from Bioline, all other nucleotides from New England Biolabs), 0.5 μM each of primers Nanog-FWD and Nanog-REV short (Sigma-Aldrich), and 1 U Phusion Hot Start II DNA Polymerase (Finnzymes). PCR was performed in a Biolabproducts Labcycler with the program 98° C./30″−[98° C./5″−60° C./10″−72° C./15″]×30−72° C./600″−12° C./∞.

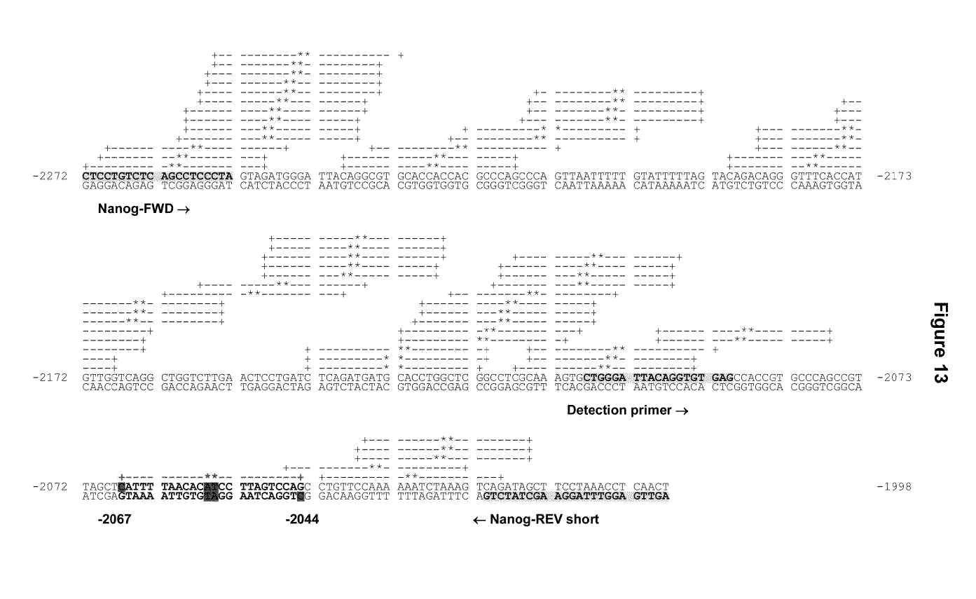

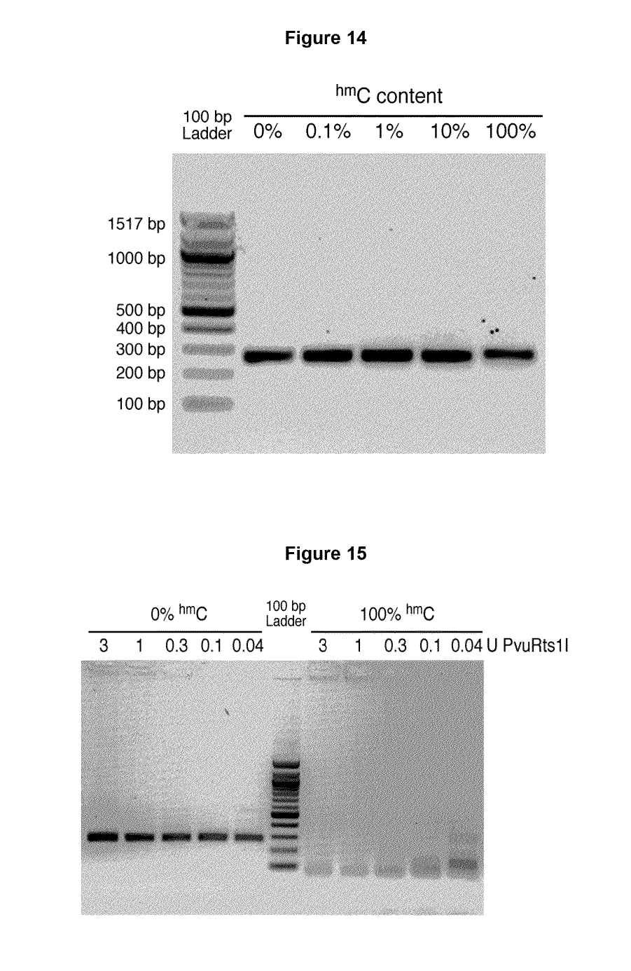

PCR fragments were purified using the GeneJET PCR Purification Kit (Fermentas), analyzed via agarose gel electrophoresis (FIG. 14), and quantified by OD₂₆₀ (Nanodrop) and fluorescence (Qubit 2.0, Life Technologies) measurements. The substrates are referred to in the following as “0% ^{hm}C”, “0.1% ^{hm}C”, “1% ^{hm}C”, “10% ^{hm}C”, and “100% ^{hm}C”.

### 2. Determination of PvuRts1I Digestion Conditions

Test digestions were performed in a total volume of 20 μI PvuRts1I reaction buffer (20 mM TrisCl pH8.0, 150 mM NaCl, 5 mM MgCl₂, 1 mM Dithiothreitol) with 100 ng DNA fragment and different concentrations of PvuRts1I at 22° C. for 15 min, followed by a heat inactivation at 65° C. for 5 min. Complete digestion of 100% ^{hm}C fragments was observed with 0.3-1 U PvuRts1I, while under no condition digestion of 0% ^{hm}C fragments could be detected (FIG. 15).

3. Second Strand Synthesis with ^{hm}C

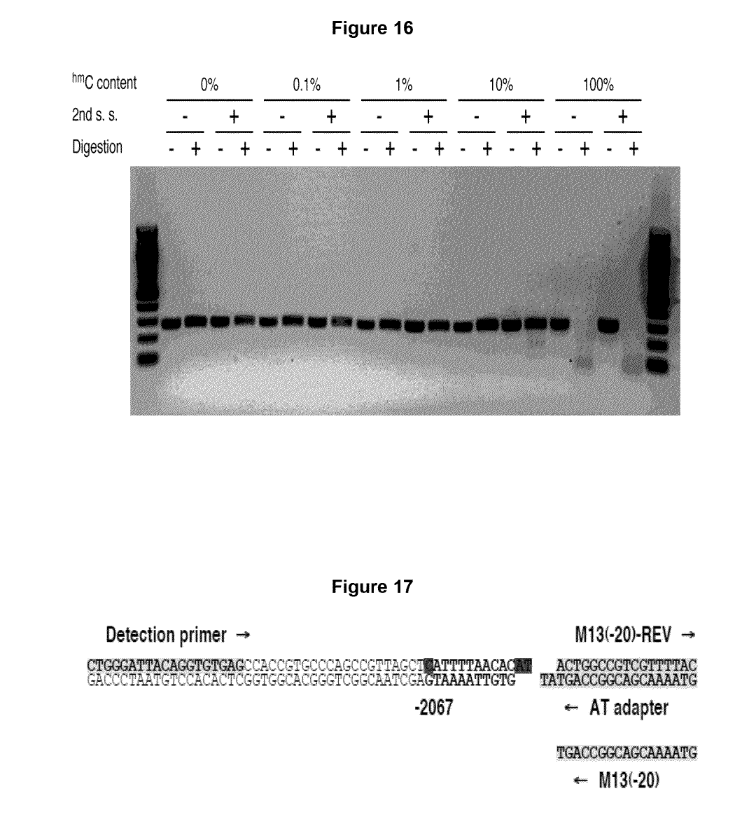

The synthesis of fully hydroxymethylated complementary strands was performed in a total volume of 50 μl 1× Phusion HF Buffer (Finnzymes) with 1 μg of each of the five substrates (0%, 0.1%, 1%, 10%, 100% ^{hm}C) as template, 200 μM each of dATP, dTTP, dGTP, and d^{hm}CTP, 0.5 μM each of primers Nanog-FWD and Nanog-REV short, and 1 U Phusion Hot Start II DNA Polymerase. The reaction was performed in a Biolabproducts Labcycler with the program 98° C./120″−60° C./60″−72° C./600″−12° C./∞. PCR fragments were purified using the GeneJET PCR Purification Kit, analyzed via agarose gel electrophoresis (FIG. 16), and quantified by OD₂₆₀ and fluorescence measurements. These substrates are referred to in the following as “0% ^{hm}C 2ss”, “0.1% ^{hm}C 2ss”, “1% ^{hm}C 2ss”, “10% ^{hm}C 2ss”, and “100% ^{hm}C 2ss”.

### 4. PvuRts1I Digestion of Substrates

Substrate digestions were performed in a total volume of 40 μl PvuRts1I reaction buffer (20 mM TrisCl pH8.0, 150 mM NaCl, 5 mM MgCl₂, 1 mM Dithiothreitol) with 200 ng DNA fragment and 1 U PvuRts1I at 22° C. for 15 min, followed by a heat inactivation at 65° C. for 5 min. 10 μl from each digestion reaction were analyzed by agarose gel electrophoresis (FIG. 16).

### 5. Adapter Ligation

The digested fragments were ligated to a adapter containing an AT 3′ overhang, generated by annealing the primers AT adapter (5′-GTA AAA CGA CGG CCA GTA T-3′) (SEQ ID NO: 4) and M13(-20)-REV (5′-ACT GGC CGT CGT TTT AC-3′) (SEQ ID NO: 5). FIG. 17 shows the 71 bp hmC detection ptoduct (SEQ ID NO: 6).

The ligation reaction was carried out in 10 μl Quick Ligation buffer (New England Biolabs) using 5 ng of digested fragment, 1.5 nmol of the adapter and additionally 0.5 μl Quick Ligase (New England Biolabs) for 5 min at 25° C., followed by heat inactivation for 5 min at 65° C.

### 6. Quantitative Detection of Ligation Products Via Real Time PCR

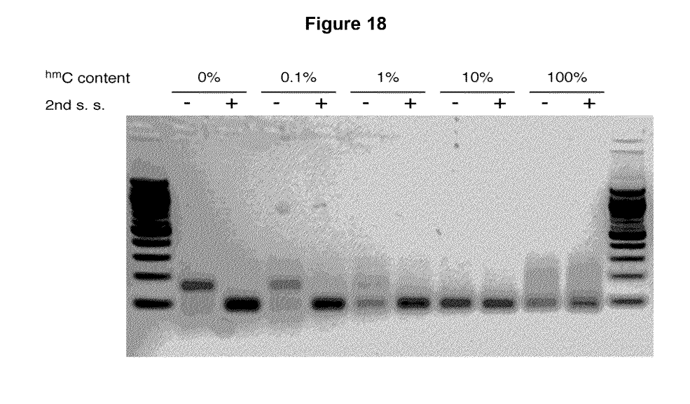

For quantitative detection of ligation products a real time PCR was performed using the Detection primer (5′-CTG GGA TTA CAG GTG TGA G-3′) (SEQ ID NO: 7) and the primer M13(-20) (5′-GTA AAA CGA CGG CCA GT-3′; FIG. 17) (SEQ ID NO: 8). The reaction volume was 20 μl with 10 μl 2× Fast SYBR Green Master Mix (Applied Biosystems), 2 μl of the ligation reaction (approximately 1 ng), and 50 μM of each primer in a CFX-96 Real-Time Cycler (BioRad) with the program 95° C./20″−[95° C./3″−60° C./30″]×0 followed by a melting curve from 65° C. to 95° C. All amplifications were performed in four technical replicates. For quality control after the run all four replicates were combined (80 and 15 μl of that analyzed by agarose gel electrophoresis (FIG. 18).

### 7. Result

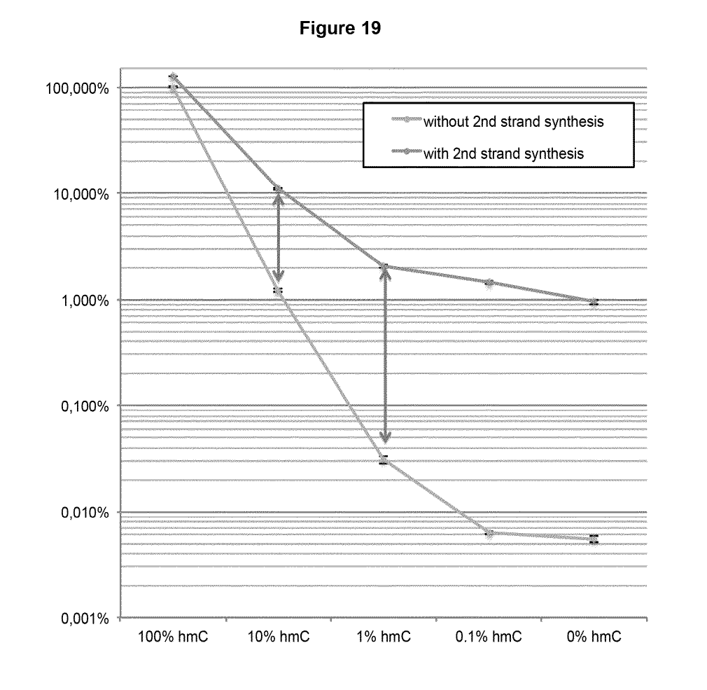

By synthesizing the complementary second strand in the presence of ^{hm}C, hemimodified recognition sites for the endonuclease PvuRts1I are converted to fully modified sites. These sites can be cut by PvuRts1I and provide template for the adapter ligation, which in turn is the template for the detection amplification. The results of this test experiment clearly show the proposed effect (double arrows in FIG. 19). Note that the upper graph shows the result with 2^{nd} strand synthesis, while the lower graph shows the result without 2^{nd} strand synthesis. Error bars indicate standard deviation.

### REFERENCES

1. Bird, A. (2002) DNA methylation patterns and epigenetic memory. Genes Dev, 16, 6-21
2. Rottach, A., Leonhardt, H. and Spada, F. (2009) DNA methylation-mediated epigenetic control. Journal of Cellular Biochemistry, 108, 43-51.
3. Kriaucionis, S, and Heintz, N. (2009) The Nuclear DNA Base 5-Hydroxymethylcytosine Is Present in Purkinje Neurons and the Brain. Science, 324, 929-930.
4. Tahiliani, M., Koh, K. P., Shen, Y., Pastor, W. A., Bandukwala, H., Brudno, Y., Agarwal, S., Iyer, L. M., Liu, D. R., Aravind, L. et al. (2009) Conversion of 5-Methylcytosine to 5-Hydroxymethylcytosine in Mammalian DNA by MLL Partner TETI. Science, 324, 930-935.
5. Ito, S., D'Alessio, A. C., Taranova, O. V., Hong, K., Sowers, L. C. and Zhang, Y. (2010) Role of Tet proteins in 5mC to 5hmC conversion, ES-cell self-renewal and inner cell mass specification. Nature, 466, 1129-1133.
6. Feng, J., Zhou, Y., Campbell, S. L., Le, T., Li, E., Sweatt, J. D., Silva, A. J. and Fan, G. (2010) Dnmt1 and Dnmt3a maintain DNA methylation and regulate synaptic function in adult forebrain neurons. Nat Neurosci, 13, 423-430.
7. Szwagierczak, A., Bultmann, S., Schmidt, C. S., Spada, F. and Leonhardt, H. (2010)

Sensitive enzymatic quantification of 5-hydroxymethylcytosine in genomic DNA. Nucleic Acids Research, 38, e181.
8. Münzel, M., Globisch, D., Bruckl, T., Wagner, M., Welzmiller, V., Michalakis, S., Muller, M., Biel, M. and Carell, T. (2010) Quantification of the Sixth DNA Base Hydroxymethylcytosine in the Brain. Angew Chem Int Ed Engl, 49, 5375-5377.
9. Globisch, D., Munzel, M., Muller, M., Michalakis, S., Wagner, M., Koch, S., Bruckl, T., Biel, M. and Carell, T. (2010) Tissue distribution of 5-hydroxymethylcytosine and search for active demethylation intermediates. PLoS ONE, 5, e15367.
10. Huang, Y., Pastor, W. A., Shen, Y., Tahiliani, M., Liu, D. R. and Rao, A. (2010) The Behaviour of 5-Hydroxymethylcytosine in Bisulfite Sequencing. PLoS ONE, 5, e8888.
11. Jin, S.-G., Kadam, S, and Pfeifer, G. P. (2010) Examination of the specificity of DNA methylation profiling techniques towards 5-methylcytosine and 5-hydroxymethylcytosine. Nucl. Acids Res., 38, e125.
12. Nestor, C., Ruzov, A., Meehan, R. and Dunican, D. (2010) Enzymatic approaches and bisulfite sequencing cannot distinguish between 5-methylcytosine and 5-hydroxymethylcytosine in DNA. Biotechniques, 48, 317-319.
13. Song, C.-X., Szulwach, K. E., Fu, Y., Dai, Q., Yi, C., Li, X., Li, Y., Chen, C.-H., Zhang, W., Jian, X. et al. (2010) Selective chemical labeling reveals the genome-wide distribution of 5-hydroxymethylcytosine. Nat Biotechnol, 29, 68-72.
14. Flaks, J. G. and Cohen, S. S. (1957) The enzymic synthesis of 5-hydroxymethyldeoxycytidylic acid. Biochim Biophys Acta, 25, 667-668.
15. Wiberg, J. S, and Buchanan, J. M. (1964) Studies on Labile Deoxycytidylate

Hydroxymethylases from Escherichia Coli B Infected with Temperature-Sensitive Mutants of Bacteriophage T4. Proc Natl Aced Sci USA, 51, 421-428.
16. Lehman, I. R. and Pratt, E. A. (1960) On the structure of the glucosylated hydroxymethylcytosine nucleotides of coliphages T2, T4, and T6. J Biol Chem, 235, 3254-3259.
17. Raleigh, E. A. (1992) Organization and function of the mcrBC genes of Escherichia coli K-12. Mol Microbiol, 6, 1079-1086.
18. Zheng, Y., Cohen-Karni, D., Xu, D., Chin, H. G., Wilson, G., Pradhan, S, and Roberts, R. J. (2010) A unique family of Mrr-like modification-dependent restriction endonucleases. Nucleic Acids Research, 38, 5527-5534.
19. Bair, C. L. and Black, L. W. (2007) A Type IV Modification Dependent Restriction Nuclease that Targets Glucosylated Hydroxymethyl Cytosine Modified DNAs. Journal of Molecular Biology, 366, 768-778.
20. Janosi, L., Yonemitsu, H., Hong, H. and Kaji, A. (1994) Molecular Cloning and Expression of a Novel Hydroxymethylcytosine-specific Restriction Enzyme (PvuRts1I) Modulated by Glucosylation of DNA. Journal of Molecular Biology, 242, 45-61.
21. Tsumura, A., Hayakawa, T., Kumaki, Y., Takebayashi, S., Sakaue, M., Matsuoka, C., Shimotohno, K., Ishikawa, F., Li, E., Ueda, H. R. et al. (2006) Maintenance of self-renewal ability of mouse embryonic stem cells in the absence of DNA methyltransferases Dnmt1, Dnmt3a and Dnmt3b. Genes Cells, 11, 805-814.
22. Crooks, G. E., Hon, G., Chandonia, J. M. and Brenner, S. E. (2004) WebLogo: a sequence logo generator. gGenome Res, 14, 1188-1190.
23. Hattori, N., Imao, Y., Nishino, K., Hattori, N., Ohgane, J., Yagi, S., Tanaka, S, and Shiota, K. (2007) Epigenetic regulation of Nanog gene in embryonic stem and trophoblast stem cells. Genes to Cells, 12, 387-396.
24. Koh, K. P., Yabuuchi, A., Rao, S., Huang, Y., Cunniff, K., Nardone, J., Laiho, A., Tahiliani, M., Sommer, C. A., Mostoslaysky, G. et al. (2011) Tet1 and Tet2 Regulate 5-Hydroxymethylcytosine Production and Cell Lineage Specification in Mouse Embryonic Stem Cells. Cell Stem Cell, 8, 200-213.
25. Okano, M., Xie, S, and Li, E. (1998) Dnmt2 is not required for de novo and maintenance methylation of viral DNA in embryonic stem cells. Nucleic Acids Res, 26, 2536-2540.
26. Yoder, J. A. and Bestor, T. H. (1998) A candidate mammalian DNA methyltransferase related to pmtl p of fission yeast. Hum Mol Genet, 7, 279-284.
27. Goll, M. G., Kirpekar, F., Maggert, K. A., Yoder, J. A., Hsieh, C. L., Zhang, X., Golic, K. G., Jacobsen, S. E. and Bestor, T. H. (2006) Methylation of tRNAAsp by the DNA methyltransferase homolog Dnmt2. Science, 311, 395-398.
28. Rai, K., Chidester, S., Zavala, C. V., Manos, E. J., James, S. R., Karpf, A. R., Jones, D. A. and Cairns, B. R. (2007) Dnmt2 functions in the cytoplasm to promote liver, brain, and retina development in zebrafish. Genes Dev., 21, 261-266.
29. Schaefer, M. and Lyko, F. (2010) Lack of evidence for DNA methylation of Invader4 retroelements in Drosophila and implications for Dnmt2-mediated epigenetic regulation. Nat Genet, 42, 920-921; author reply 921.
30. Schaefer, M. and Lyko, F. (2010) Solving the Dnmt2 enigma. Chromosoma, 119, 35-40.
31. Zemach, A., McDaniel, I. E., Silva, P. and Zilberman, D. (2010) Genome-wide evolutionary analysis of eukaryotic DNA methylation. Science, 328, 916-919.
32. Gou, D., Rubalcava, M., Sauer, S., Mora-Bermudez, F., Erdjument-Bromage, H., Tempst, P., Kremmer, E. and Sauer, F. (2010) SETDBI Is Involved in Postembryonic DNA Methylation and Gene Silencing in Drosophila. PLoS ONE, 5, e10581.
33. Phalke, S., Nickel, 0., Walluscheck, D., Hortig, F., Onorati, M. C. and Reuter, G. (2009) Retrotransposon silencing and telomere integrity in somatic cells of Drosophila depends on the cytosine-5 methyltransferase DNMT2. Nat Genet, 41, 696-702.

1. A method of detecting a hydroxymethyl (hm) cytosine (C) in a nucleic acid molecule preparation or determining or evaluating the hydroxymethylation status within a nucleic acid molecule preparation; comprising:
(a) providing a single-stranded (ss) nucleic acid molecule;
(b) synthesizing at least one copy of at least a portion of the complementary strand of said ss nucleic acid molecule thereby generating a double-stranded (ds) nucleic acid molecule, wherein said synthesis is carried out in the presence of hydroxymethylcytosine or analog thereof (e.g., protected hydroxyl group); and
(c) reacting the product obtained in (b) with an endonuclease being capable of cleaving said ds nucleic acid molecule, wherein cleavage by said endonuclease requires a recognition site that contains hmC on opposite strands; and
(d) analyzing the product obtained in step (c).

2. (canceled)

3. The method of claim 1 performed for determining or evaluating the hydroxymethylation status of a subject.

4. The method of claim 2 performed for diagnosing a disease in the subject, said disease being characterized by an aberrant hydroxymethylation status wherein step
(a) comprises providing a sample obtained from said subject, said sample comprising the single-stranded (ss) nucleic acid molecule.

5. The method of claim 1, wherein all of the product obtained in step (b) or a purified product obtained in step (b) is reacted with said endonuclease.

6. The method of claim 1, wherein step (d) comprises
(i) sequencing,
(ii) PCR, preferably qPCR, and/or
(iii) primer extension.

7. The method of claim 1, wherein said nucleic acid molecule is genomic DNA (gDNA) or mitochondrial DNA (mtDNA).

8. The method of claim 4, wherein said disease is a neurodegenerative disease.

9. The method of claim 4, wherein said disease is an age-related disease.

10. The method of claim 9, wherein said age-related disease is selected from the group consisting of cardiovascular disease, cancer, arthritis, cataract, osteoporosis, type 2 diabetes, and hypertension.

11. The method of claim 1, wherein said endonuclease is one or more selected from PvuRtsII, PpeHI, EsaSS310P, EsaRBORFBP, PatTI, Ykrl, EsaNI, SpeAI, BbiDI, PfrCORFlI80P, PcoORF314P, BmeDI, AbaSDFI, AbaCI, AbaAI, AbaSI, AbaUMB30RFAP, Asp60RFAP and/or catalytically active mutants and derivatives thereof.

12. The method of claim 1, wherein said endonuclease is an endonuclease of the PvuRts1I family.

13. The method of claim 1, further comprising comparing the results obtained in step (d) with a reference sample.

14. A kit for performing the method of claim 1 comprising hmC and an endonuclease of the PvuRts1I family.

15. The kit of claim 14, wherein said endonuclease of the PvuRts1I family is PvuRts 1I.

16. The kit of claim 14 which is a diagnostic kit.

17. A composition comprising (a) PvuRts1I and (b1) about 10% glycerol and 1 mM DTT or (b2) a reaction buffer having a ionic strength that is equal to or above the ionic strength of about 150 mM NaCl.

18. (canceled)

19. The composition of claim 17, wherein PvuRts1I has cleavage activity on a nucleic acid molecule, in particular on DNA at the sequence ^{hm}CN₁₁₋₁₂/N₉₋₁₀G (SEQ ID NO:27), whereby cleavage results in two nucleotides 3′ overhang.
